(19) United States
(12) Patent Application Publication
(10) Pub. No.: US 2024/0365662 A1
(43) Pub. Date: Oct. 31, 2024

(54) PHOTOACTIVE NONFULLERENE ACCEPTORS OF THE A-D-A'-D-A TYPE FOR USE IN OPTOELECTRONIC DEVICES

(71) Applicant: SUMITOMO CHEMICAL CO., LTD., Tokyo (JP)

(72) Inventors: Michal Maciejczyk, Godmanchester (GB); Nir Yaacobi-Gross, Godmanchester (GB); Florence Bourcet, Godmanchester (GB); Ben Gardner, Godmanchester (GB); Yuki Yokoi, Godmanchester (GB)

(73) Assignee: SUMITOMO CHEMICAL CO., LTD., Tokyo (JP)

(21) Appl. No.: 18/681,320

(22) PCT Filed: Aug. 5, 2022
(86) PCT No.: PCT/EP2022/072160

§ 371 Date: Feb. 5, 2024

(30) Foreign Application Priority Data
Aug. 6, 2021 (GB) 2111398.0
Mar. 24, 2022 (GB) 2204177.6
Mar. 24, 2022 (GB) 2204179.2
Jun. 7, 2022 (GB) 2208329.9

(51) Int. Cl. H10K 85/60; C07D 519/00; C08G 61/12; H10K 30/30; H10K 85/10

(52) U.S. Cl. CPC H10K 85/657; C07D 519/00; C08G 61/126; H10K 85/113; H10K 85/151; H10K 85/626; H10K 85/655; H10K 85/6576; C08G 2261/124; C08G 2261/1412; C08G 2261/149; C08G 2261/18; C08G 2261/228; C08G 2261/324; C08G 2261/3246; C08G 2261/94; H10K 30/30

(57) ABSTRACT

A compound of formula (I), D¹ and D² are electron-donating groups; A² and A³ are electron-accepting groups; B¹ and B² are bridging groups; x1 and x2 are 0, 1, 2 or 3; y1 and y2 are at least 1; z1 and z2 are 0, 1, 2 or 3; and A¹ is a group of formula (II) wherein Ar¹ is an aromatic or heteroaromatic group; and Y is O, S, NR⁴ or R1-C═C—R¹ wherein R¹ in each occurrence is independently H or a substituent wherein two substituents R¹ may be linked to form a monocyclic or polycyclic ring and R⁴ is H or a substituent. The compound may be used as an electron-accepting material with an electron-donating material in an organic photodetector.

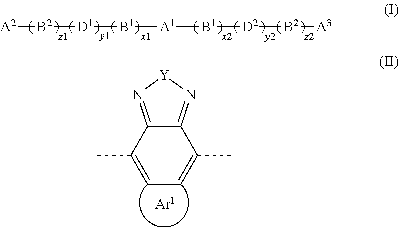

### BACKGROUND

Embodiments of the present disclosure relate to electron-accepting compounds and more specifically, but not by way of limitation, to compounds containing electron-accepting and electron donating units, the compounds being suitable for use as an electron-accepting material in a photoresponsive device.

Electron-accepting non-fullerene compounds are known.

Yoon et al, “Effects of Electron Donating and Electron-Accepting Substitution on Photovoltaic Performance in Benzothiadiazole-Based A-D-A′-D-A-Type Small-Molecule Acceptor Solar Cells” ACS Appl. Energy Mater. 2020, 3, 12, 12327-12337 discloses A-D-A′-D-A-type acceptors for use in solar cells.

Gao et al, “Non-fullerene acceptors with nitrogen-containing six-membered heterocycle cores for the applications in organic solar cells” Solar Energy Materials and Solar Cells 225, 2021, 111046 discloses non-fullerene acceptors with pyrazine or pyridazine as the cores.

Wang et al, “Near-infrared absorbing non-fullerene acceptors with unfused D-A-D core for efficient organic solar cells” Organic Electronics 92, 2021, 106131 discloses a D-A-D core employing 3-bis(4-(2-ethylhexyl)-thiophen-2-yl)-5,7-bis(2ethylhexyl)benzo-[1,2:4,5-c′]-dithiophene-4,8-dione (BDD) unit as the A moiety and 4,4-dialkyl-4H-cyclopenta[2,1-b:3,4-b′]dithiophene (CPDT) unit as the D moiety.

CN110379926 discloses an organic solar cell based on a benzodithiazole near-infrared receptor.

CN112608333 discloses a small molecule based on a bisthiadiazole carbazole derivative.

CN112259687 discloses a ternary fullerene organic solar cell.

### SUMMARY

In some embodiments, the present disclosure provides a compound of formula (I):

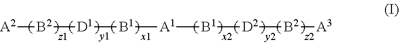

wherein:
D¹ and D² independently in each occurrence is an electron-donating group;
A² and A³ are each independently an electron-accepting group;
B¹ and B² in each occurrence are independently a bridging group;
x1 and x2 are each independently 0, 1, 2 or 3;
y1 and y2 are each independently at least 1;
z1 and z2 are each independently 0, 1, 2 or 3; and
A¹ is a group of formula (II):

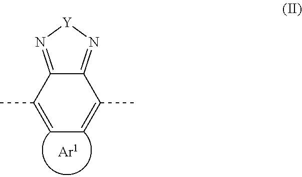

wherein:
Ar¹ is an aromatic or heteroaromatic group; and
Y is O, S, NR⁴ or R¹—C═C—R¹ wherein R¹ in each occurrence is independently H or a substituent wherein two substituents R¹ may be linked to form a monocyclic or polycyclic ring and R⁴ is H or a substituent.

Optionally, the group of formula (II) has formula (IIa):

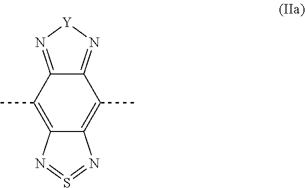

Optionally, the group of formula (II) has formula (IIb):

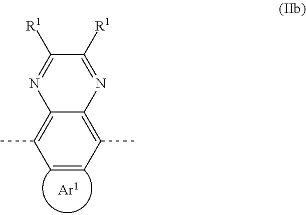

In some embodiments, the two R¹ groups are not linked. According to these embodiments, optionally each R¹ is independently selected from H; F; CN; NO₂; C₁₋₂₀ alkyl wherein one or more non-adjacent C atoms may be replaced with O, S, CO, COO, NR⁴, PR⁴, or Si(R³)₂ and one or more H atoms may be replaced with F; and aryl or heteroaryl which may be unsubstituted or substituted with one or more substituents, wherein R³ and R⁴ are each independently H or a substituent.

In some embodiments, the two R¹ groups are linked. According to these embodiments, optionally the compound of formula (IIb) has formula (IIb-1) or (IIb-2):

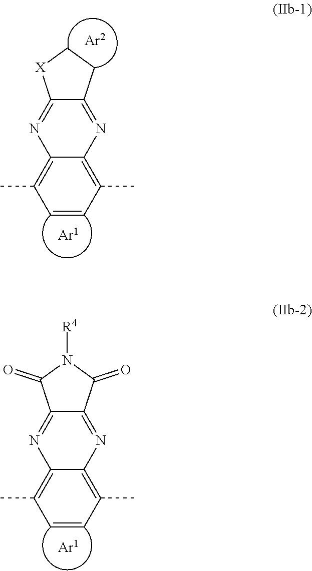

wherein
Ar² is an aromatic or heteroaromatic group which is unsubstituted or substituted with one or more substituents; and
X is selected from O, S, SO₂, NR⁴, PR⁴, C(R³)₂, Si(R³)₂ C═O, C═S and C═C(R⁵)₂ wherein R³ and R⁴ independently in each occurrence are selected from H and a substituent and R⁵ independently in each occurrence is an electron-withdrawing group.

Optionally, Ar² is benzene which is unsubstituted or substituted with one or more substituents.

Optionally, at least one of x1 and x2 is at least 1 and B¹ in each occurrence is independently selected from vinylene, arylene, heteroarylene, arylenevinylene and heteroarylenevinylene, each of which is unsubstituted or substituted with one or more substituents.

Optionally, at least one of z1 and z2 is at least 1 and B² in each occurrence is independently selected from vinylene, arylene, heteroarylene, arylenevinylene and heteroarylenevinylene, each of which is unsubstituted or substituted with one or more substituents.

Optionally, D¹ and D² are each independently selected from units of formulae (VIIa)-(VIIp) as described herein.

Optionally, at least one of A² and A³ comprises a non-aromatic carbon-carbon double bond and a carbon atom of the carbon-carbon double bond is bound directly to D¹ or D² or, if present, to B².

Optionally, A² and A³ are each independently selected from groups of formulae (IIIa)-(IIIq) as described herein.

Optionally, at least one A is a group of formula (IIIa-1):

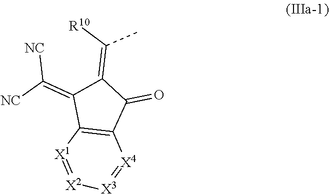

wherein:
each X¹—X⁴ is independently CR¹² or N wherein R¹² in each occurrence is H or a substituent selected from C₁₋₂₀ hydrocarbyl and an electron-withdrawing group and wherein R¹⁰ is H or a substituent.

Optionally, the polymer has an absorption peak of greater than 900 nm.

According to some embodiments, the present disclosure provides a compound of formula (I):

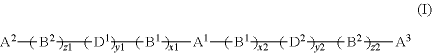

wherein:
A¹ is an electron-accepting group;
D¹ and D² independently in each occurrence is an electron-donating group;
A¹, A² and A³ are each independently an electron-accepting group;
B¹ and B² in each occurrence are independently a bridging group;
x1 and x2 are each independently 0, 1, 2 or 3;
y1 and y2 are each independently at least 1;
z1 and z2 are each independently 0, 1, 2 or 3;
and at least one of (i)-(iv) applies:
(D¹)_{y1} and (D²)_{y2} are different;
A² and A³ are different;
(B¹)ₓ₁ and (B¹)ₓ₂ are different; and
(B²)_{z1} and (B²)_{z2} are different.

D¹, D², A¹, A², A³, B¹, B², x1, x2, y1, y2, z1 and z2 according to these embodiments may be as described anywhere herein.

In some embodiments, the present disclosure provides a compound of formula (X):

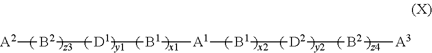

wherein:
A¹, A² and A³ are each independently an electron-accepting group;
D¹ and D² independently in each occurrence is an electron-donating group;
B¹ and B² independently in each occurrence is a bridging group;
x1 and x2 are each independently 0, 1, 2 or 3;
y1 and y2 are each independently at least 1; and
z³ and z⁴ are each independently 0, 1, 2 or 3 with the proviso that at least one of z³ and z⁴ is at least 1.

D¹, D², A¹, A², A³, B¹, B², x1, x2, y1, y2, z1 and z2 of formula (X) may be as described anywhere herein, for example as described with respect to compounds of formula (I).

In some embodiments, the present disclosure provides composition comprising an electron-donating material and an electron-accepting material wherein the electron accepting material is a compound as described herein.

In some embodiments, the present disclosure provides an organic electronic device comprising an active layer comprising a compound or composition as described herein.

Optionally, the organic electronic device is an organic photoresponsive device comprising a bulk heterojunction layer disposed between an anode and a cathode and wherein the bulk heterojunction layer comprises a composition as described herein.

Optionally, the organic photoresponsive device is an organic photodetector.

In some embodiments, the present disclosure provides a photosensor comprising a light source and an organic photodetector as described herein, wherein the photosensor is configured to detect light emitted from the light source.

Optionally, the light source emits light having a peak wavelength of greater than 900 nm.

In some embodiments, the present disclosure provides a formulation comprising a compound or composition as described herein dissolved or dispersed in one or more solvents.

In some embodiments, the present disclosure provides a method of forming an organic electronic device as described herein wherein formation of the active layer comprises deposition of a formulation according as described herein onto a surface and evaporation of the one or more solvents.

### DESCRIPTION OF DRAWINGS

The disclosed technology and accompanying figures describe some implementations of the disclosed technology.

FIG. 1 illustrates an organic photoresponsive device according to some embodiments; and

FIG. 2A shows absorption spectra for films of Compound Examples 1 and 2 a compound according to embodiments of the present disclosure and Comparative Compound 1;

FIG. 2B shows absorption spectra for films of Compound Examples 1 and 2 a compound according to embodiments of the present disclosure and Comparative Compound 2;

FIG. 3A shows an absorption spectrum for a film of Compound Example 3;

FIG. 3B shows a long wavelength range detail of the spectrum of FIG. 3A;

FIG. 3C shows an absorption spectrum for a solution of Compound Example 3;

FIG. 3D shows a long wavelength range detail of the spectrum of FIG. 3C;

FIG. 4A shows an absorption spectrum for a film of Compound Example 4;

FIG. 4B shows a long wavelength range detail of the spectrum of FIG. 4A;

FIG. 4C shows an absorption spectrum for a solution of Compound Example 4;

FIG. 4D shows a long wavelength range detail of the spectrum of FIG. 4C;

FIG. 5 shows absorption spectra for o-dichlorobenzene solutions of Compound Examples 5, 6 and 7;

FIG. 6 shows absorption spectra in o-dichlorobenzene for Compound Examples 6 and 8;

FIG. 7 shows absorption spectra in o-dichlorobenzene for Compound Example 9 and a comparative compound;

FIG. 8 is a graph of external quantum efficiencies between 500 and 1700 nm for an organic photodetector containing Compound Example 1 according to an embodiment of the present disclosure;

FIG. 9A is a graph of current density vs. reverse bias voltage for an organic photodetector containing Compound Example 3 according to an embodiment of the present disclosure under dark conditions and under illumination at 940 nm, 1200 nm and white light;

FIG. 9B is a graph of external quantum efficiency vs. wavelength at −2V applied voltage for the device of FIG. 6A;

FIG. 10 is a graph of external quantum efficiency vs wavelength at a −5V applied voltage for an organic photodetector containing Compound Example 4 according to an embodiment of the present disclosure; and

FIG. 11 is graphs of external quantum efficiency vs wavelength at a −5V applied voltage for organic photodetectors containing Compound Example 5, 6 and 7 according to an embodiment of the present disclosure.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers are may be present. References to a layer “on” another layer when used in this application means that the layers are in direct contact. References to a specific atom include any isotope of that atom unless specifically stated otherwise.

The teachings of the technology provided herein can be applied to other systems, not necessarily the system described below. The elements and acts of the various examples described below can be combined to provide further implementations of the technology.

Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

A compound of formula (I) or (X) as described herein may be provided in a bulk heterojunction layer of a photoresponsive device, preferably a photodetector, in which the bulk heterojunction layer is disposed between an anode and a cathode.

The bulk heterojunction layer comprises or consists of an electron-donating material and an electron-accepting compound of formula (I) or (X) as described herein.

In some embodiments, the bulk heterojunction layer contains two or more accepting materials and/or two or more electron-accepting materials.

In some embodiments, the weight of the electron-donating material(s) to the electron-accepting material(s) is from about 1:0.5 to about 1:2, preferably about 1:1.1 to about 1:2.

Preferably, the electron-donating material has a type II interface with the electron-accepting material, i.e. the electron-donating material has a shallower HOMO and LUMO that the corresponding HOMO and LUMO levels of the electron-accepting material. Preferably, the compound of formula (I) or (X) has a HOMO level that is at least 0.05 eV deeper, optionally at least 0.10 eV deeper, than the HOMO of the electron-donating material.

Optionally, the gap between the HOMO level of the electron-donating material and the LUMO level of the electron-accepting compound of formula (I) or (X) is less than 1.4 eV.

Unless stated otherwise, HOMO and LUMO levels of materials as described herein are as measured by square wave voltammetry (SWV).

In SWV, the current at a working electrode is measured while the potential between the working electrode and a reference electrode is swept linearly in time. The difference current between a forward and reverse pulse is plotted as a function of potential to yield a voltammogram. Measurement may be with a CHI 660D Potentiostat.

The apparatus to measure HOMO or LUMO energy levels by SWV may comprise a cell containing 0.1 M tertiary butyl ammonium hexafluorophosphate in acetonitrile; a 3 mm diameter glassy carbon working electrode; a platinum counter electrode and a leak free Ag/AgCl reference electrode.

Ferrocene is added directly to the existing cell at the end of the experiment for calculation purposes where the potentials are determined for the oxidation and reduction of ferrocene versus Ag/AgCl using cyclic voltammetry (CV).

The sample is dissolved in toluene (3 mg/ml) and spun at 3000 rpm directly on to the glassy carbon working electrode.

LUMO=4.8-E ferrocene (peak to peak average)−E reduction of sample (peak maximum).

HOMO=4.8-E ferrocene (peak to peak average)+E oxidation of sample (peak maximum).

A typical SWV experiment runs at 15 Hz frequency; 25 mV amplitude and 0.004 V increment steps. Results are calculated from 3 freshly spun film samples for both the HOMO and LUMO data.

In some embodiments, the compound of formula (I) or (X) has an absorption peak greater than 900 nm, optionally greater than 1000 nm, optionally greater than 1200 nm.

Unless stated otherwise, absorption spectra of materials as described herein are measured using a Cary 5000 UV-VIS-NIR Spectrometer. Measurements were taken from 175 nm to 3300 nm using a PbSmart NIR detector for extended photometric range with variable slit widths (down to 0.01 nm) for optimum control over data resolution.

Absorption data are obtained by measuring the intensity of transmitted radiation through a solution sample. Absorption intensity is plotted vs. incident wavelength to generate an absorption spectrum. A method for measuring film absorption may comprise measuring a 15 mg/ml solution in a quartz cuvette and comparing to a cuvette containing the solvent only.

Unless stated otherwise, absorption data as provided herein is as measured in toluene solution.

In some embodiments, the electron-accepting compound has formula (I):

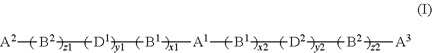

D¹ and D² independently in each occurrence is an electron-donating group.
A′, A² and A³ are each independently an electron-accepting group.
B¹ and B² in each occurrence are independently a bridging group.
x1 and x2 are each independently 0, 1, 2 or 3, preferably 0 or 1.
y1 and y2 are each independently at least 1, preferably 1, 2 or 3, more preferably 1.
z1 and z2 are each independently 0, 1, 2 or 3, preferably 0 or 1.

Each of the electron-accepting groups A¹, A² and A³ has a lowest unoccupied molecular orbital (LUMO) level that is deeper (i.e., further from vacuum) than the LUMO of either of the electron-donating groups D¹ or D², preferably at least 1 eV deeper. The LUMO levels of electron-accepting groups and electron-donating groups may be as determined by modelling the LUMO level of these groups, in which each bond to adjacent group is replaced with a bond to a hydrogen atom. Modelling may be performed using Gaussian09 software available from Gaussian using Gaussian09 with B3LYP (functional) and LACVP* (Basis set).

In some embodiments, A¹ of formula (I) is a group of formula (II):

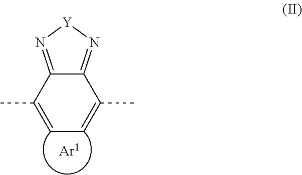

wherein:
Ar¹ is an aromatic or heteroaromatic group; and
Y is O, S, NR⁴ or R¹—C═C—R¹ wherein R¹ in each occurrence is independently H or a substituent wherein two substituents R¹ may be linked to form a monocyclic or polycyclic ring; and R⁴ is H or a substituent.

In some embodiments the compound of formula (I) is a “symmetric” compound in which —(B¹)x1-(D¹)y1-(B²)z1-A² is the same as —(B¹)x2-(D²)y2-(B²)z2-A³.

In some embodiments the compound of formula (I) is a compound in which —(B′)x1-(D¹)y1-(B²)z1-A² is different from —(B¹)x2-(D²)y2-(B²)z2-A³. Such compounds are described hereinafter as “asymmetric” compounds.

In an asymmetric compound of formula (I), at least one of (i)-(iv) applies:
(i) (D¹)_{y1} and (D²)_{y2} are different;
(ii) A² and A³ are different;
(iii) (B¹)ₓ₁ and (B¹)ₓ₂ are different; and
(iv) (B²)_{z1} and (B²)_{z2} are different.

Optionally, D¹ and D² are different and y1 and y2 are the same or different.

Optionally, y1 and y2 are different and D¹ and D² are the same or different.

If x1 and x2 are the same or different and are both greater than 1 then optionally B¹ of (B¹)ₓ₁ is different from B¹ of (B)ₓ₂

Optionally, x1 and x2 are different.

If z1 and z2 are the same or different and are both greater than 1 then optionally B² of (B²)_{z1} is different from B² of (B²)_{z2}.

Optionally, z1 and z2 are different.

In some embodiments, the present disclosure provides compounds of formula (X):

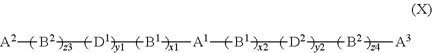

wherein A¹, A², A³, B¹, B², D¹, D², x1, x2, y1 and y2 are as described above with respect to formula (I) and z3 and z4 are each independently 0, 1, 2 or 3 with the proviso that at least one of z3 and z4 is at least 1.

### Acceptor Unit A¹

In the case where A¹ is a group of formula (II), Ar¹ may be a monocyclic or polycyclic heteroaromatic group which is unsubstituted or substituted with one or more R² groups wherein R² in each occurrence is independently a substituent.

Preferred R² groups are selected from
F;
CN;
NO₂;
C₁₋₂₀ alkyl wherein one or more non-adjacent C atoms may be replaced with O, S, NR⁷ wherein R⁷ is a C₁₋₁₂ hydrocarbyl, COO or CO and one or more H atoms of the alkyl may be replaced with F;
an aromatic or heteroaromatic group, preferably phenyl, which is unsubstituted or substituted with one or more substituents; and
a group selected from

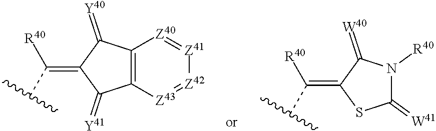

wherein Z⁴⁰, Z⁴¹, Z⁴² and Z⁴³ are each independently CR¹³ or N wherein R¹³ in each occurrence is H or a substituent, preferably a C₁₋₂₀ hydrocarbyl group; Y⁴⁰ and Y⁴¹ are each independently O, S, NX⁷¹ wherein X⁷¹ is CN or COOR⁴⁰; or CX⁶⁰X⁶¹ wherein X⁶⁰ and X⁶¹ is independently CN, CF₃ or COOR⁴⁰; W⁴⁰ and W⁴¹ are each independently O, S, NX⁷¹ or CX⁶⁰X⁶¹ wherein X⁶⁰ and X⁶¹ is independently CN, CF₃ or COOR⁴⁰; and R⁴⁰ in each occurrence is H or a substituent, preferably H or a C₁₋₂₀ hydrocarbyl group. Exemplary substituents of an aromatic or heteroaromatic group R² are F, CN, NO₂, and C₁₋₁₂ alkyl wherein one or more non-adjacent C atoms may be replaced with O, S, NR⁷, COO or CO and one or more H atoms of the alkyl may be replaced with F.

R⁷ as described anywhere herein may be, for example, C₁₋₁₂ alkyl, unsubstituted phenyl; or phenyl substituted with one or more C₁₋₆ alkyl groups.

If a C atom of an alkyl group as described anywhere herein is replaced with another atom or group, the replaced C atom may be a terminal C atom of the alkyl group or a non-terminal C-atom.

By “non-terminal C atom” of an alkyl group as used anywhere herein means a C atom other than the C atom of the methyl group at the end of an n-alkyl chain or the C atoms of the methyl groups at the ends of a branched alkyl chain.

If a terminal C atom of a group as described anywhere herein is replaced then the resulting group may be an anionic group comprising a countercation, e.g., an ammonium or metal countercation, preferably an ammonium or alkali metal cation.

A C atom of an alkyl substituent group which is replaced with another atom or group as described anywhere herein is preferably a non-terminal C atom, and the resultant substituent group is preferably non-ionic.

Exemplary monocyclic heteroaromatic groups Ar¹ are oxadiazole, thiadiazole, triazole and 1,4-diazine which is unsubstituted or substituted with one or more substituents. Thiadiazole is particularly preferred.

Exemplary polycyclic heteroaromatic groups Ar¹ are groups of formula (V):

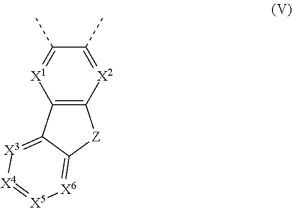

X¹ and X², are each independently selected from N and CR³ wherein R³ is H or a substituent, optionally H or a substituent R² as described above.

X³, X⁴, X⁵ and X⁶ are each independently selected from N and CR³ with the proviso that at least one of X³, X⁴, X⁵ and X⁶ is CR³.

Z is selected from O, S, SO₂, NR⁴, PR⁴, C(R³)₂, Si(R³)₂ C═O, C═S and C═C(R⁵)₂ wherein R³ is as described above; R⁴ is H or a substituent; and R⁵ in each occurrence is an electron-withdrawing group.

Optionally, each R⁴ of any NR⁴ or PR⁴ described anywhere herein is independently selected from H; C₁₋₂₀ alkyl wherein one or more non-adjacent C atoms other than the C atom bound to N or P may be replaced with O, S, NR⁷, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms of the alkyl may be replaced with O, S, NR⁷, COO or CO and one or more H atoms of the alkyl may be replaced with F.

Preferably, each R⁵ is CN, COOR⁴⁰; or CX⁶⁰X⁶¹ wherein X⁶⁰ and X⁶¹ is independently CN, CF₃ or COOR⁴⁰ and R⁴⁰ in each occurrence is H or a substituent, preferably H or a C₁₋₂₀ hydrocarbyl group.

A¹ groups of formula (II) are preferably selected from groups of formulae (IIa) and (IIb):

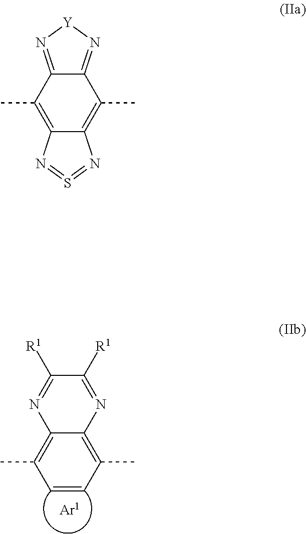

For compounds of formula (IIb), the two R¹ groups may or may not be linked.

Preferably, when the two R¹ groups are not linked each R¹ is independently selected from H; F; CN; NO₂; C₁₋₂₀ alkyl wherein one or more non-adjacent C atoms may be replaced with O, S, NR₇, CO, COO, NR⁴, PR⁴, or Si(R³)₂ wherein R³ and R⁴ are as described above and one or more H atoms may be replaced with F; and aryl or heteroaryl, preferably phenyl, which may be unsubstituted or substituted with one or more substituents. Substituents of the aryl or heteroaryl group may be selected from one or more of F; CN; NO₂; and C₁₋₂₀ alkyl wherein one or more non-adjacent C atoms may be replaced with O, S, NR⁷, CO, COO and one or more H atoms may be replaced with F.

Preferably, when the two R¹ groups are linked, the group of formula (IIb) has formula (IIb-1) or (IIb-2):

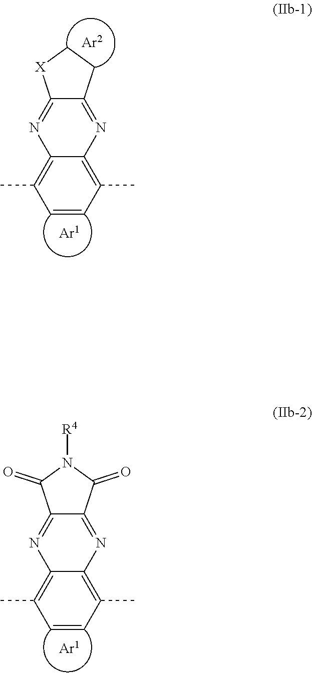

Ar² is an aromatic or heteroaromatic group, preferably benzene, which is unsubstituted or substituted with one or more substituents. Ar² may be unsubstituted or substituted with one or more substituents R² as described above.

X is selected from O, S, SO₂, NR⁴, PR⁴, C(R³)₂, Si(R³)₂ C═O, C═S and C═C(R⁵)₂ wherein R³, R⁴ and R⁵ are as described above.

Exemplary electron-accepting groups of formula (II) include, without limitation:

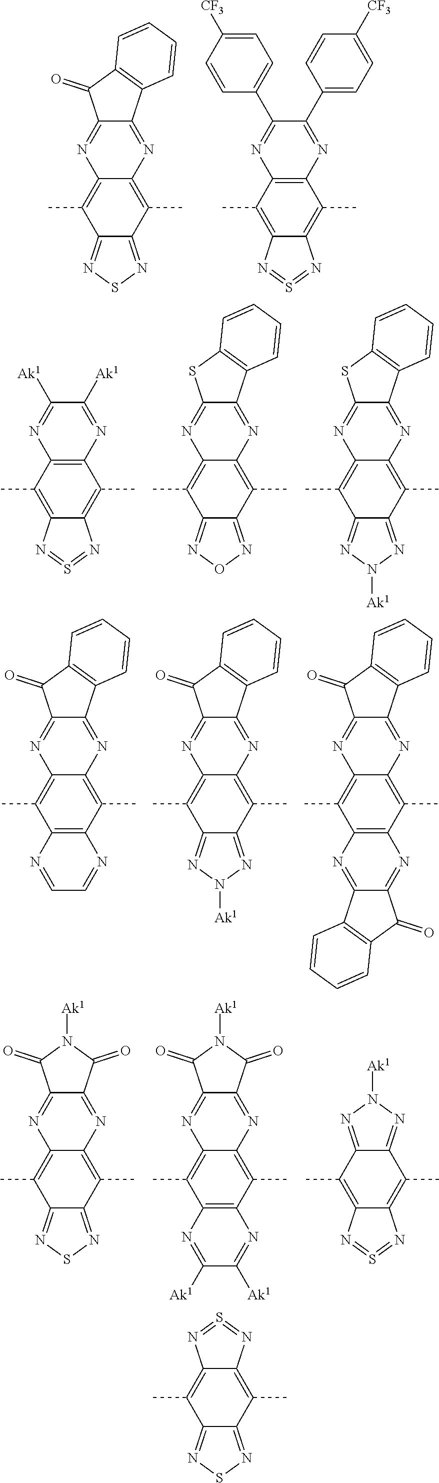

wherein Ak¹ is a C₁₋₂₀ alkyl group

Divalent electron-accepting groups other than formula (II) are optionally selected from formulae (IVa)-(IVj)

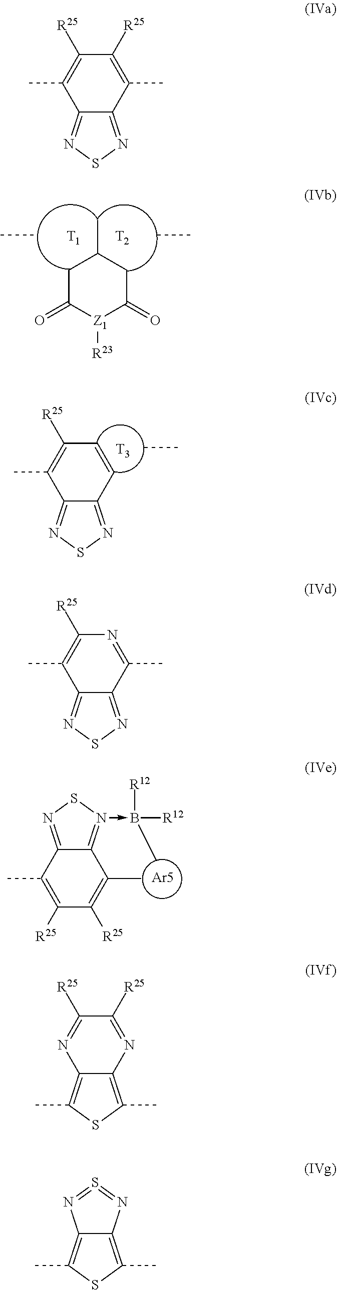

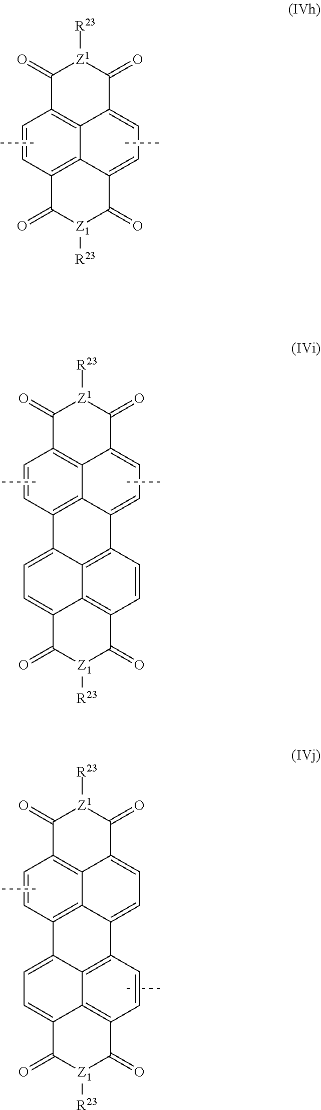

R²³ in each occurrence is a substituent, optionally C₁₋₁₂ alkyl wherein one or more non-adjacent C atoms other than the C atom attached to Z¹ may be replaced with O, S, NR⁷, COO or CO and one or more H atoms of the alkyl may be replaced with F.

R²⁵ in each occurrence is independently H; F; CN; NO₂; C₁₋₁₂ alkyl wherein one or more non-adjacent C atoms may be replaced with O, S, NR⁷, COO or CO and one or more H atoms of the alkyl may be replaced with F; an aromatic group, optionally phenyl, which is unsubstituted or substituted with one or more substituents selected from F and C₁₋₁₂ alkyl wherein one or more non-adjacent C atoms may be replaced with O, S, NR⁷, COO or CO; or

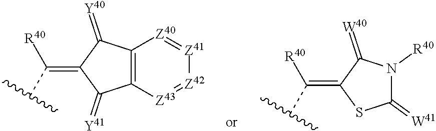

wherein Z⁴⁰, Z⁴¹, Z⁴² and Z⁴³ are each independently CR¹³ or N wherein R¹³ in each occurrence is H or a substituent, preferably a C₁₋₂₀ hydrocarbyl group;
Y⁴⁰ and Y⁴¹ are each independently O, S, NX⁷¹ wherein X⁷¹ is CN or COOR⁴⁰; or CX⁶⁰X⁶¹ wherein X⁶⁰ and X⁶¹ is independently CN, CF₃ or COOR⁴⁰;
W⁴⁰ and W⁴¹ are each independently O, S, NX⁷¹ wherein X⁷¹ is CN or COOR⁴⁰; or CX⁶⁰X⁶¹ wherein X⁶⁰ and X⁶¹ is independently CN, CF₃ or COOR⁴⁰; and
R⁴⁰ in each occurrence is H or a substituent, preferably H or a C₁₋₂₀ hydrocarbyl group.
Z¹ is N or P.

T¹, T² and T³ each independently represent an aryl or a heteroaryl ring, optionally benzene, which may be fused to one or more further rings. Substituents of T¹, T² and T³, where present, are optionally selected from non-H groups of R²⁵.

R¹² in each occurrence is a substituent, preferably a C₁₋₂₀ hydrocarbyl group.

Ar⁵ is an arylene or heteroarylene group, optionally thiophene, fluorene or phenylene, which may be unsubstituted or substituted with one or more substituents, optionally one or more non-H groups selected from R²⁵.

Electron-Accepting Groups A², A³

The monovalent acceptor Groups A² and A³ may each independently be selected from any such units known to the skilled person. A² and A³ may be the same or different, preferably different.

Exemplary monovalent acceptor units include, without limitation, units of formulae (IIIa)-(IIIq)

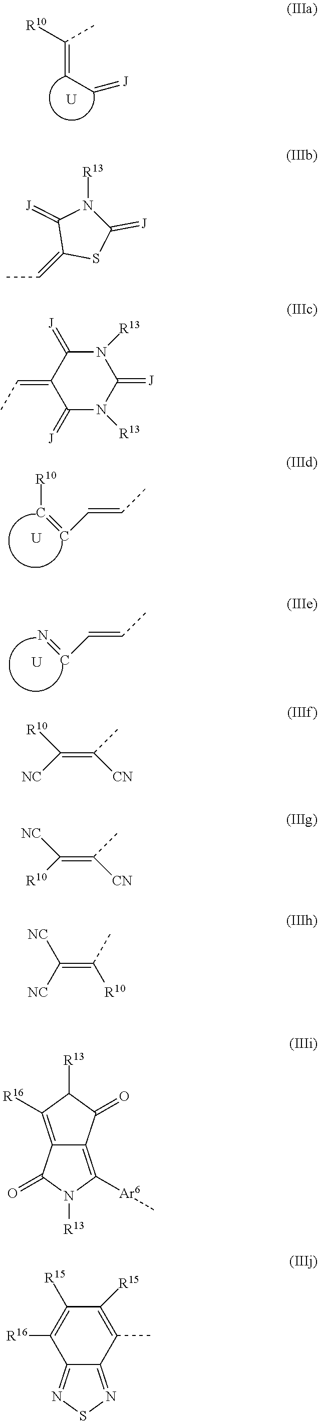

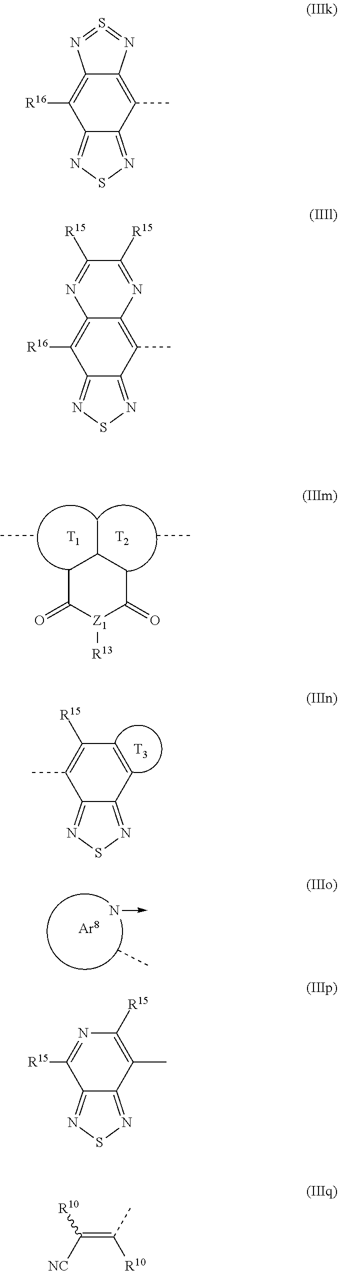

U is a 5- or 6-membered ring which is unsubstituted or substituted with one or more substituents and which may be fused to one or more further rings.

The N atom of formula (IIIe) may be unsubstituted or substituted.

R¹⁰ is H or a substituent, preferably a substituent selected from the group consisting of C₁₋₁₂ alkyl wherein one or more non-adjacent C atoms may be replaced with O, S, NR⁷, COO or CO and one or more H atoms of the alkyl may be replaced with F; and an aromatic group, optionally phenyl, which is unsubstituted or substituted with one or more substituents selected from F and C₁₋₁₂ alkyl wherein one or more non-adjacent C atoms may be replaced with O, S, NR⁷, COO or CO.

Preferably, R¹⁰ is H.

J is O or S, preferably O.

R¹³ in each occurrence is a substituent, optionally C₁₋₁₂ alkyl wherein one or more non-adjacent C atoms may be replaced with O, S, NR⁷, COO or CO and one or more H atoms of the alkyl may be replaced with F.

R¹⁵ in each occurrence is independently H; F; C₁₋₁₂ alkyl wherein one or more non-adjacent C atoms may be replaced with O, S, NR⁷, COO or CO and one or more H atoms of the alkyl may be replaced with F; aromatic group Ar², optionally phenyl, which is unsubstituted or substituted with one or more substituents selected from F and C₁₋₁₂ alkyl wherein one or more non-adjacent C atoms may be replaced with O, S, NR⁷, COO or CO; or a group selected from:

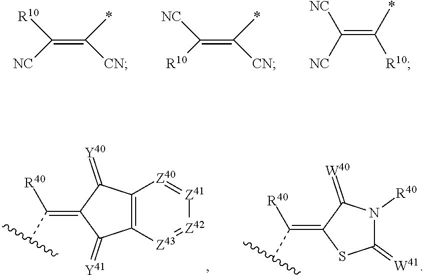

R¹⁶ is H or a substituent, preferably a substituent selected from:
—(Ar³)_{w} wherein Ar³ in each occurrence is independently an unsubstituted or substituted aryl or heteroaryl group, preferably thiophene, and w is 1, 2 or 3;

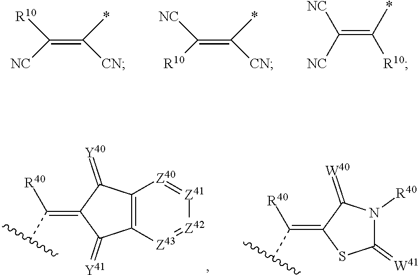

and
C₁₋₁₂ alkyl wherein one or more non-adjacent C atoms may be replaced with O, S, NR⁷, COO or CO and one or more H atoms of the alkyl may be replaced with F.

Ar⁶ is a 5-membered heteroaromatic group, preferably thiophene or furan, which is unsubstituted or substituted with one or more substituents.

Substituents of Ar³ and Ar⁶, where present, are optionally selected from C₁₋₁₂ alkyl wherein one or more non-adjacent C atoms may be replaced with O, S, NR⁷, COO or CO and one or more H atoms of the alkyl may be replaced with F.

T¹, T² and T³ are each independently as described above.

Ar⁸ is a fused heteroaromatic group which is unsubstituted or substituted with one or more substituents, optionally one or more non-H substituents R¹⁰, and which is bound to an aromatic C atom of B² and to a boron substituent of B².

Preferred groups A² and A³ are groups having a non-aromatic carbon-carbon bond which is bound directly to D¹ or D² or, if present to B2.

Preferably at least one of A² and A³, preferably both of A² and A³, are a group of formula (IIIa-1):

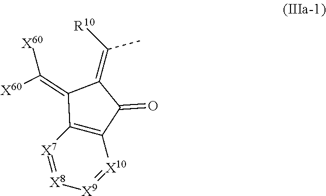

wherein:
R¹⁰ is as described above;
each X⁷—X¹⁰ is independently CR¹² or N wherein R¹² in each occurrence is H or a substituent selected from C₁₋₂₀ hydrocarbyl and an electron withdrawing group. Preferably, the electron withdrawing group is F, Cl, Br or CN, more preferably F, Cl or CN; and
X⁶⁰ and X⁶¹ is independently CN, CF₃ or COOR⁴⁰ wherein R⁴⁰ in each occurrence is H or a substituent, preferably H or a C₁₋₂₀ hydrocarbyl group. Preferably, X⁶⁰ and X⁶¹ are each CN.

The C₁₋₂₀ hydrocarbyl group R¹² may be selected from C₁₋₂₀ alkyl; unsubstituted phenyl; and phenyl substituted with one or more C₁₋₁₂ alkyl groups.

Exemplary groups of formula (IIId) include:

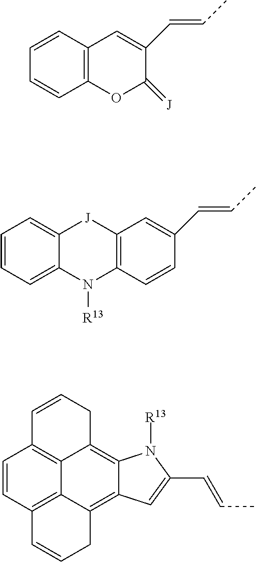

Exemplary groups of formula (IIIe) include:

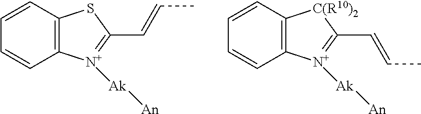

An exemplary group of formula (IIIq) is:

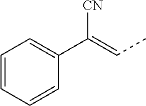

An exemplary group of formula (IIIg) is:

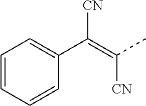

An exemplary group of formula (IIIj) is:

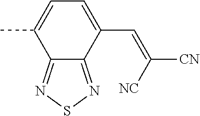

wherein Ak is a C₁₋₁₂ alkylene chain in which one or more C atoms may be replaced with O, S, NR⁷, CO or COO; An is an anion, optionally —SO₃—; and each benzene ring is independently unsubstituted or substituted with one or more substituents selected from substituents described with reference to R¹⁰.

Exemplary groups of formula (IIIm) are:

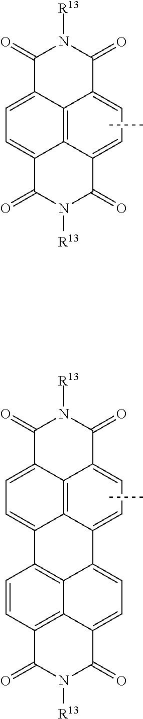

An exemplary group of formula (IIIn) is:

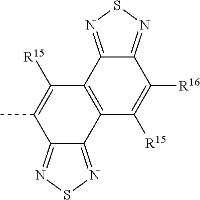

Groups of formula (IIIo) are bound directly to a bridging group B² substituted with a —B(R^{M})₂ wherein R¹⁴ in each occurrence is a substituent, optionally a C₁₋₂₀ hydrocarbyl group; → is a bond to the boron atom —B(R¹⁴)₂ of R³ or R⁶; and --- is the bond to B².

Optionally, R¹⁴ is selected from C₁₋₁₂ alkyl; unsubstituted phenyl; and phenyl substituted with one or more C₁₋₁₂ alkyl groups.

The group of formula (IIIo), the B² group and the B(R¹⁴)₂ substituent of B² may be linked together to form a 5- or 6-membered ring.

Optionally groups of formula (IIIo) are selected from:

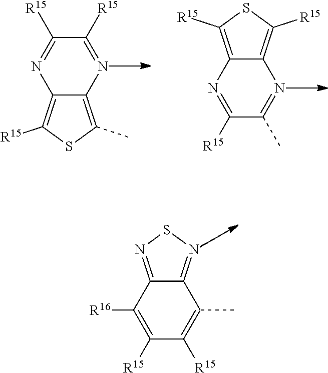

### Bridging Units

Bridging units B¹ and B² are preferably each selected from vinylene, arylene, heteroarylene, arylenevinylene and heteroarylenevinylene wherein the arylene and heteroarylene groups are monocyclic or bicyclic groups, each of which may be unsubstituted or substituted with one or more substituents.

Bridging units B¹ and B² preferably are monocyclic or fused bicyclic arylene or heteroarylene groups, more preferably monocyclic or fused bicyclic heteroarylene groups.

Optionally, B¹ and B² are is selected from units of formulae (VIa)-(VIg):

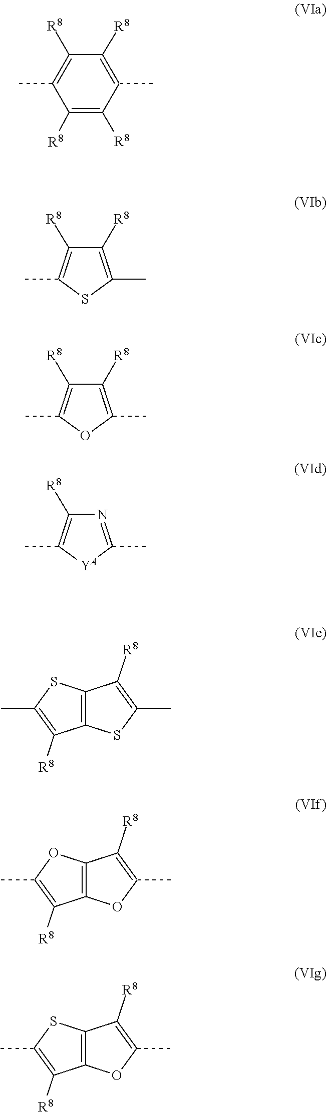

wherein Y^{A} is O, S or NR⁵ wherein R⁵⁵ is H or a substituent; R⁸ in each occurrence is independently H or a substituent, preferably H or a substituent selected from F; CN; NO₂; C₁₋₂₀ alkyl wherein one or more non-adjacent C atoms may be replaced with O, S, NR⁷, COO or CO and one or more H atoms of the alkyl may be replaced with F; phenyl which is unsubstituted or substituted with one or more substituents; and —B(R¹⁴)₂ wherein R¹⁴ in each occurrence is a substituent, optionally a C₁₋₂₀ hydrocarbyl group. R⁸ groups of formulae (VIa), (VIb) and (VIc) may be linked to form a bicyclic ring, for example thienopyrazine.

R⁸ is preferably H, C₁₋₂₀ alkyl or C₁₋₁₉ alkoxy.

Electron-Donating Groups D¹ and D²

Electron-donating groups preferably are fused aromatic or heteroaromatic groups, more preferably fused heteroaromatic groups containing 3 or more rings. Particularly preferred electron-donating groups comprise fused thiophene or furan rings, optionally fused rings containing thiophene or furan rings and one or more rings selected from benzene, cyclopentadiene, tetrahydropyran, tetrahydrothiopyran and piperidine rings, each of said rings being unsubstituted or substituted with one or more substituents.

Exemplary electron-donating groups D¹ and D² include groups of formulae (VIIa)-(VIIp):

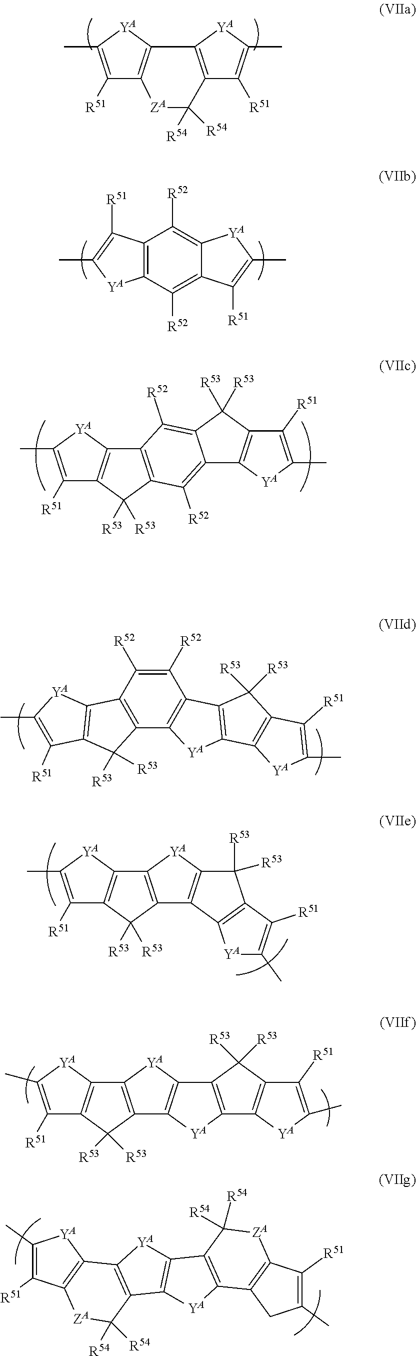

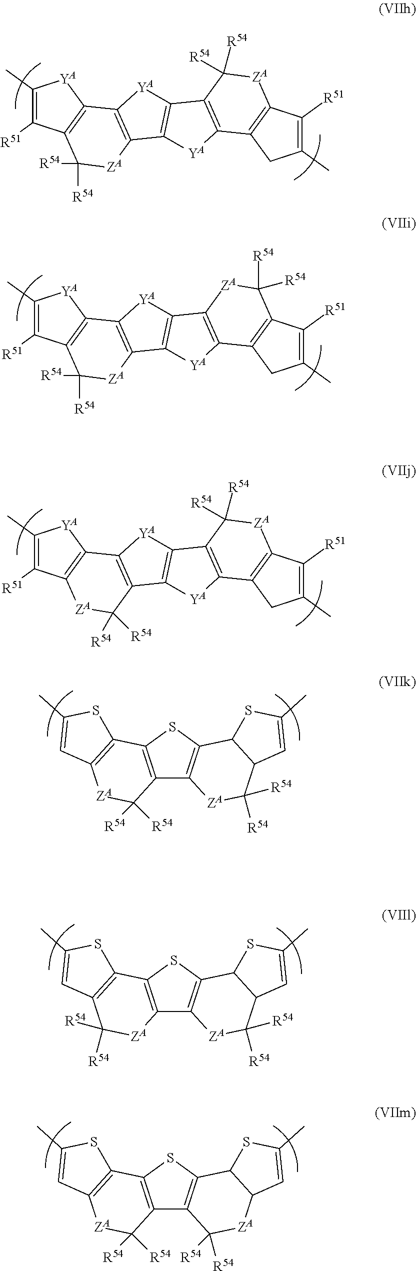

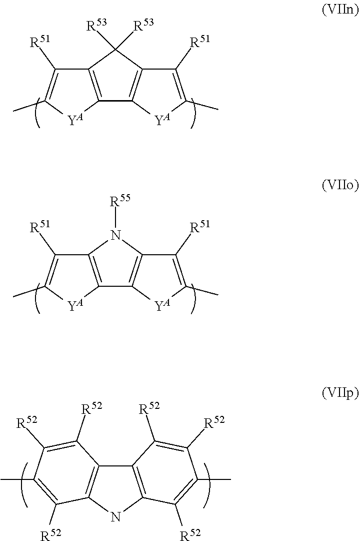

wherein Y^{A} in each occurrence is independently O, S or NR⁵, Z^{A} in each occurrence is O, CO, S, NR⁵⁵ or C(R⁵⁴)₂; R⁵¹, R⁵² R⁵⁴ and R⁵⁵ independently in each occurrence is H or a substituent; and R⁵³ independently in each occurrence is a substituent.

Optionally, R⁵¹ and R⁵² independently in each occurrence are selected from H; F; C₁₋₂₀ alkyl wherein one or more non-adjacent C atoms may be replaced with O, S, NR⁷, COO or CO and one or more H atoms of the alkyl may be replaced with F; and an aromatic or heteroaromatic group Ar³ which is unsubstituted or substituted with one or more substituents.

In some embodiments, Ar³ may be an aromatic group, e.g., phenyl.

The one or more substituents of Ar³, if present, may be selected from C₁₋₁₂ alkyl wherein one or more non-adjacent C atoms may be replaced with O, S, NR⁷, COO or CO and one or more H atoms of the alkyl may be replaced with F.

Preferably, each R⁵⁴ is selected from the group consisting of:
H;
F;
linear, branched or cyclic C₁₋₂₀ alkyl wherein one or more non-adjacent C atoms may be replaced by O, S, NR⁷, CO or COO wherein R⁷ is a C₁₋₁₂ hydrocarbyl and one or more H atoms of the C₁₋₂₀ alkyl may be replaced with F; and
a group of formula (Ak)u-(Ar⁷)v wherein Ak is a C₁₋₂₀ alkylene chain in which one or more non-adjacent C atoms may be replaced with O, S, NR⁷, CO or COO; u is 0 or 1; Ar⁷ in each occurrence is independently an aromatic or heteroaromatic group which is unsubstituted or substituted with one or more substituents; and v is at least 1, optionally 1, 2 or 3.

Substituents of Ar⁷, if present, are preferably selected from F; Cl; NO₂; CN; and C₁₋₂₀ alkyl wherein one or more non-adjacent C atoms may be replaced with O, S, NR⁷, CO or COO and one or more H atoms may be replaced with F. Preferably, Ar⁷ is phenyl.

Preferably, each R⁵¹ is H.

Optionally, R⁵³ independently in each occurrence is selected from C₁₋₂₀ alkyl wherein one or more non-adjacent C atoms may be replaced with O, S, NR⁷, COO or CO and one or more H atoms of the alkyl may be replaced with F; and phenyl which is unsubstituted or substituted with one or more substituents, optionally one or more C₁₋₁₂ alkyl groups wherein one or more non-adjacent C atoms may be replaced with O, S, NR⁷, COO or CO and one or more H atoms of the alkyl may be replaced with F.

Preferably, R⁵⁵ as described anywhere herein is H or C₁₋₃₀ hydrocarbyl group.

Preferably, D¹ and D² are each independently a group of formula (VIIa). Exemplary groups of formula (VIIa) include, without limitation:

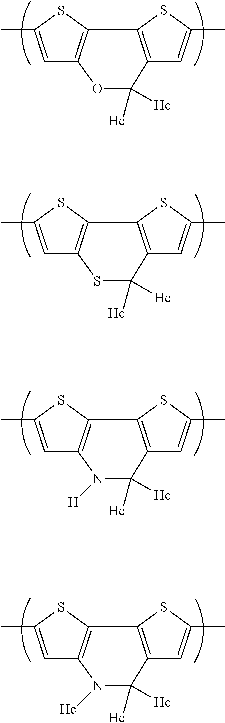

wherein He in each occurrence is independently a C₁₋₂₀ hydrocarbyl group, e.g., C₁₋₂₀ alkyl, unsubstituted aryl, or aryl substituted with one or more C₁₋₁₂ alkyl groups. The aryl group is preferably phenyl.

In some embodiments, y1 and y2 are each 1.

In some embodiments, at least one of y1 and y2 is greater than 1. In these embodiments, the chain of D¹ and/or D² groups, respectively, may be linked in any orientation. For example, in the case where D¹ is a group of formula (VIIa) and y1 is 2, -[D¹]_{y1}-may be selected from any of:

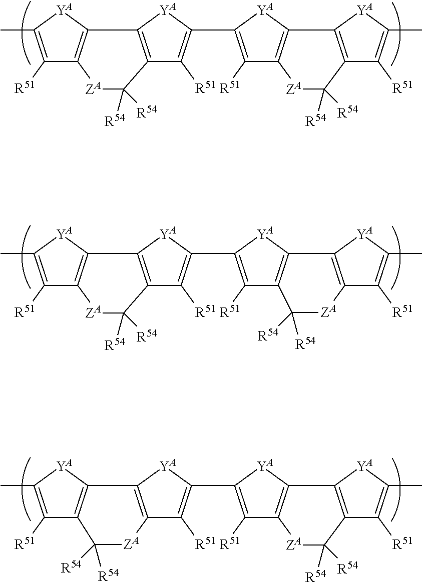

### Electron-Donating Material

A bulk heterojunction layer as described herein comprises an electron-donating material and a compound of formula (I) or (X) as described herein.

Exemplary donor materials are disclosed in, for example, WO2013051676, the contents of which are incorporated herein by reference.

The electron-donating material may be a non-polymeric or polymeric material.

In a preferred embodiment the electron-donating material is an organic conjugated polymer, which can be a homopolymer or copolymer including alternating, random or block copolymers. The conjugated polymer is preferably a donor-acceptor polymer comprising alternating electron-donating repeat units and electron-accepting repeat units.

Preferred are non-crystalline or semi-crystalline conjugated organic polymers.

Further preferably the electron-donating polymer is a conjugated organic polymer with a low bandgap, typically between 2.5 eV and 1.5 eV, preferably between 2.3 eV and 1.8 eV. Optionally, the electron-donating polymer has a HOMO level no more than 5.5 eV from vacuum level. Optionally, the electron-donating polymer has a HOMO level at least 4.1 eV from vacuum level. As exemplary electron-donating polymers, polymers selected from conjugated hydrocarbon or heterocyclic polymers including polyacene, polyaniline, polyazulene, polybenzofuran, polyfluorene, polyfuran, polyindenofluorene, polyindole, polyphenylene, polypyrazoline, polypyrene, polypyridazine, polypyridine, polytriarylamine, poly(phenylene vinylene), poly(3-substituted thiophene), poly(3,4-bisubstituted thiophene), polyselenophene, poly(3-substituted selenophene), poly(3,4-bisubstituted selenophene), poly(bisthiophene), poly(terthiophene), poly(bisselenophene), poly(terselenophene), polythieno[2,3-b]thiophene, polythieno[3,2-b]thiophene, polybenzothiophene, polybenzo[1,2-b:4,5-b′jdithiophene, polyisothianaphthene, poly(monosubstituted pyrrole), poly(3,4-bisubstituted pyrrole), poly-1,3,4-oxadiazoles, polyisothianaphthene, derivatives and co-polymers thereof may be mentioned.

Preferred examples of donor polymers are copolymers of polyfluorenes and polythiophenes, each of which may be substituted, and polymers comprising benzothiadiazole-based and thiophene-based repeating units, each of which may be substituted.

A particularly preferred donor polymer comprises donor unit (VIIa) provided as a repeat unit of the polymer, most preferably with an electron-accepting repeat unit, for example divalent electron-accepting units as described herein provided as polymeric repeat units.

### Additional Electron-Accepting Materials

In some embodiments, the compound of formula (I) or (X) as described herein is the only electron-accepting material of a bulk heterojunction layer.

In some embodiments, the bulk heterojunction layer contains a compound of formula (I) or (X) and one or more further electron-accepting materials. The one or more further electron-accepting materials may be selected from non-fullerene acceptors and fullerenes.

Non-fullerene acceptors are described in, for example, Cheng et. al., “Next-generation organic photovoltaics based on non-fullerene acceptors”, Nature Photonics volume 12, pages 131-142 (2018), the contents of which are incorporated herein by reference, and which include, without limitation, PDI, ITIC, ITIC, IEICO and derivatives thereof, e.g., fluorinated derivatives thereof such as ITIC-4F and IEICO-4F.

Exemplary fullerene electron-accepting compounds are C₆₀, C₇₀, C₇₆, C₇₈ and C₈₄ fullerenes or a derivative thereof, including, without limitation, PCBM-type fullerene derivatives including phenyl-C₆₁-butyric acid methyl ester (C₆₀PCBM), TCBM-type fullerene derivatives (e.g. tolyl-C₆₁-butyric acid methyl ester (C₆₀TCBM)), and ThCBM-type fullerene derivatives (e.g. thienyl-C₆₁-butyric acid methyl ester (CoThCBM).

Fullerene derivatives may have formula (V):

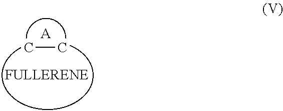

wherein A, together with the C—C group of the fullerene, forms a monocyclic or fused ring group which may be unsubstituted or substituted with one or more substituents.

Exemplary fullerene derivatives include formulae (Va), (Vb) and (Vc):

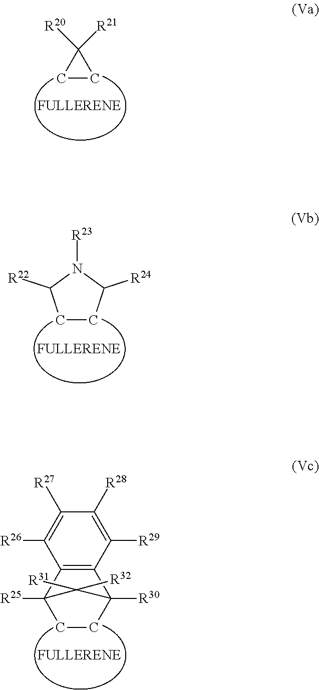

wherein R²⁰—R³² are each independently H or a substituent.

Substituents R²⁰—R³² are optionally and independently in each occurrence selected from the group consisting of aryl or heteroaryl, optionally phenyl, which may be unsubstituted or substituted with one or more substituents; and C₁₋₂₀ alkyl wherein one or more non-adjacent C atoms may be replaced with O, S, NR⁷, CO or COO and one or more H atoms may be replaced with F.

Substituents of aryl or heteroaryl, where present, are optionally selected from C₁₋₁₂ alkyl wherein one or more non-adjacent C atoms may be replaced with O, S, NR⁷, CO or COO and one or more H atoms may be replaced with F.

### Formulations

The bulk heterojunction layer may be formed by any process including, without limitation, thermal evaporation and solution deposition methods.

Preferably, the bulk heterojunction layer is formed by depositing a formulation comprising the electron-donating material(s), the electron-accepting material(s) and any other components of the bulk heterojunction layer dissolved or dispersed in a solvent or a mixture of two or more solvents. The formulation may be deposited by any coating or printing method including, without limitation, spin-coating, dip-coating, roll-coating, spray coating, doctor blade coating, wire bar coating, slit coating, ink jet printing, screen printing, gravure printing and flexographic printing.

The one or more solvents of the formulation may optionally comprise or consist of benzene substituted with one or more substituents selected from chlorine, C₁₋₁₀ alkyl and C₁₋₁₀ alkoxy wherein two or more substituents may be linked to form a ring which may be unsubstituted or substituted with one or more C₁₋₆ alkyl groups, optionally toluene, xylenes, trimethylbenzenes, tetramethylbenzenes, anisole, indane and its alkyl-substituted derivatives, and tetralin and its alkyl-substituted derivatives.

The formulation may comprise a mixture of two or more solvents, preferably a mixture comprising at least one benzene substituted with one or more substituents as described above and one or more further solvents. The one or more further solvents may be selected from esters, optionally alkyl or aryl esters of alkyl or aryl carboxylic acids, optionally a C₁₋₁₀ alkyl benzoate, benzyl benzoate or dimethoxybenzene. In preferred embodiments, a mixture of trimethylbenzene and benzyl benzoate is used as the solvent. In other preferred embodiments, a mixture of trimethylbenzene and dimethoxybenzene is used as the solvent.

The formulation may comprise further components in addition to the electron-accepting material, the electron-donating material and the one or more solvents. As examples of such components, adhesive agents, defoaming agents, deaerators, viscosity enhancers, diluents, auxiliaries, flow improvers colourants, dyes or pigments, sensitizers, stabilizers, nanoparticles, surface-active compounds, lubricating agents, wetting agents, dispersing agents and inhibitors may be mentioned.

Organic Electronic Device A polymer or composition as described herein may be provided as an active layer of an organic electronic device. In a preferred embodiment, a bulk heterojunction layer of an organic photoresponsive device, more preferably an organic photodetector, comprises a composition as described herein.

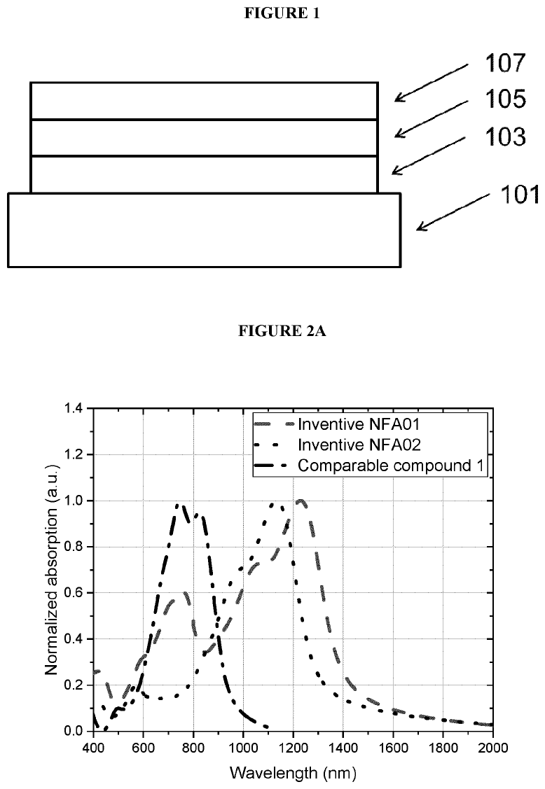

FIG. 1 illustrates an organic photoresponsive device according to some embodiments of the present disclosure. The organic photoresponsive device comprises a cathode 103, an anode 107 and a bulk heterojunction layer 105 disposed between the anode and the cathode. The organic photoresponsive device may be supported on a substrate 101, optionally a glass or plastic substrate.

Each of the anode and cathode may independently be a single conductive layer or may comprise a plurality of layers.

At least one of the anode and cathode is transparent so that light incident on the device may reach the bulk heterojunction layer. In some embodiments, both of the anode and cathode are transparent. The transmittance of a transparent electrode may be selected according to an emission wavelength of a light source for use with the organic photodetector.

FIG. 1 illustrates an arrangement in which the cathode is disposed between the substrate and the anode. In other embodiments, the anode may be disposed between the cathode and the substrate.

The organic photoresponsive device may comprise layers other than the anode, cathode and bulk heterojunction layer shown in FIG. 1. In some embodiments, a hole-transporting layer is disposed between the anode and the bulk heterojunction layer. In some embodiments, an electron-transporting layer is disposed between the cathode and the bulk heterojunction layer. In some embodiments, a work function modification layer is disposed between the bulk heterojunction layer and the anode, and/or between the bulk heterojunction layer and the cathode.

The area of the OPD may be less than about 3 cm², less than about 2 cm², less than about 1 cm², less than about 0.75 cm², less than about 0.5 cm² or less than about 0.25 cm². Optionally, each OPD may be part of an OPD array wherein each OPD is a pixel of the array having an area as described herein, optionally an area of less than 1 mm², optionally in the range of 0.5 micron²-900 micron².

The substrate may be, without limitation, a glass or plastic substrate. The substrate can be an inorganic semiconductor. In some embodiments, the substrate may be silicon. For example, the substrate can be a wafer of silicon. The substrate is transparent if, in use, incident light is to be transmitted through the substrate and the electrode supported by the substrate.

The bulk heterojunction layer contains a polymer as described herein and an electron-accepting compound. The bulk heterojunction layer may consist of these materials or may comprise one or more further materials, for example one or more further electron-donating materials and/or one or more further electron-accepting compounds.

Applications A circuit may comprise the OPD connected to a voltage source for applying a reverse bias to the device and/or a device configured to measure photocurrent. The voltage applied to the photodetector may be variable. In some embodiments, the photodetector may be continuously biased when in use.

In some embodiments, a photodetector system comprises a plurality of photodetectors as described herein, such as an image sensor of a camera.

In some embodiments, a sensor may comprise an OPD as described herein and a light source wherein the OPD is configured to receive light emitted from the light source. In some embodiments, the light source has a peak wavelength of at least 900 nm or at least 1000 nm, optionally in the range of 1000-1500 nm.

The present inventors have found that a material comprising an electron-accepting unit of formula (I) may be used for the detection of light at longer wavelengths, particularly 1300-1400 nm.

In some embodiments, the light from the light source may or may not be changed before reaching the OPD. For example, the light may be reflected, filtered, down-converted or up-converted before it reaches the OPD.

The organic photoresponsive device as described herein may be an organic photovoltaic device or an organic photodetector. An organic photodetector as described herein may be used in a wide range of applications including, without limitation, detecting the presence and/or brightness of ambient light and in a sensor comprising the organic photodetector and a light source. The photodetector may be configured such that light emitted from the light source is incident on the photodetector and changes in wavelength and/or brightness of the light may be detected, e.g., due to absorption by, reflection by and/or emission of light from an object, e.g. a target material in a sample disposed in a light path between the light source and the organic photodetector. The sample may be a non-biological sample, e.g. a water sample, or a biological sample taken from a human or animal subject. The sensor may be, without limitation, a gas sensor, a biosensor, an X-ray imaging device, an image sensor such as a camera image sensor, a motion sensor (for example for use in security applications) a proximity sensor or a fingerprint sensor. A 1D or 2D photosensor array may comprise a plurality of photodetectors as described herein in an image sensor. The photodetector may be configured to detect light emitted from a target analyte which emits light upon irradiation by the light source or which is bound to a luminescent tag which emits light upon irradiation by the light source. The photodetector may be configured to detect a wavelength of light emitted by the target analyte or a luminescent tag bound thereto.

### EXAMPLES

### Compound Example 1

Compound Example 1 (NFA1) was prepared according to the following scheme:

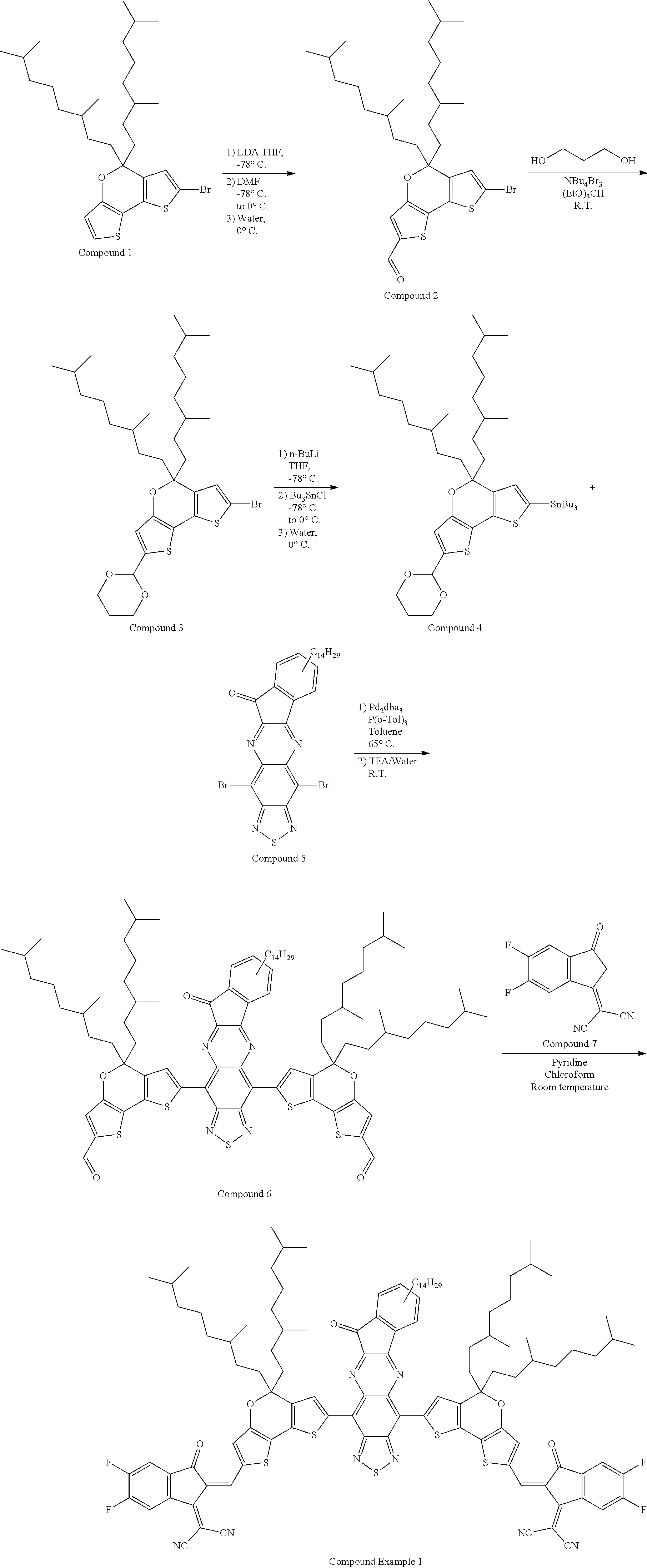

### Step 1

### Step 1

To a solution of Compound 1 (0.52 g, 0.94 mmol) in anhydrous THF (15 mL) at −85° C. was added LDA (0.79 mL, 0.99 mmol) dropwise. The mixture was stirred at −80° C. for 45 min. DMF (0.11 mL, 1.40 mmol) was added dropwise over 5 min and the mixture was stirred at below −80° C. for 20 min before being allowed to warm to room temperature. The reaction was quenched with 10 ml of 2M HCl, extracted with toluene, and theorganic phase washed twice with water, dried over magnesium sulfate and concentrated to dryness. Purification via column chromatography [elutant: 30% dichloromethane in heptane] gave Compound 2 as yellow oil (0.17 g, 31% yield)

### Step 2

Compound 2 (0.17 g, 0.29 mmol), propane-1,3-diol (0.08 ml, 1.16 mmol), tetrabutylammonium tribromide (1.4 mg, 0.003 mmol), triethyl orthoformate (0.05 mL, 0.32 mmol), p-toluenesulfonic acid monohydrate (0.07 g, 0.35 mmol) and toluene (0.4 mL) were stirred at room temperature for 1 hour, at 70° C. for 1.5 hours and then at 100° C. for 1.5 hours at. The reaction mixture was cooled to room temperature and diluted with 10 ml of toluene, and the organic phase was washed once with aqueous NaHCO₃ (15 ml), and water, and dried over magnesium sulfate and concentrated to dryness. Purification via column chromatography (elutant: 2% to 5% ethyl acetate in heptane) gave Compound 3 as yellow oil (70 mg, 38% yield).

### Step 3

To a solution of Compound 3 (0.07 g, 0.11 mmol) in anhydrous THF (7 mL) at −93° C. was added n-butyllithium (0.05 mL, 0.13 mmol) dropwise and the mixture stirred at approximately −90° C. for 15 min. Tributyltin chloride (0.04 mL, 0.14 mmol) was added dropwise and the mixture was allowed to warm up slowly to 7° C. over 4.5 hours and then cooled down to 0° C. and water (10 mL) was added slowly maintaining an internal temperature of about approximately 0° C. The mixture was extracted with toluene and the organic phase was washed twice with water, dried over magnesium sulfate and concentrated to dryness to give Compound 4, (94.3 mg 101% yield). The product was used in the next step without further purification.

### Step 4

To degassed toluene (10 ml), Compound 4 (0.09 g, 0.11 mmol) and Compound 5 (0.03 g, 0.047 mmol) were added. To this mixture tris(dibenzylideneacetone) dipalladium (0.004 g, 0.003 mmol) and tris(o-tolyl)phosphine (0.003 g, 0.014 mmol) were added, the mixture was further degassed for 5 min and was then heated to 70° C. for 30 minutes. The temperature was increased to 100° C. and the mixture was stirred for 1 hour. The reaction mixture was cooled to room temperature, water (10 mL) was added and the mixture was stirred for 5 minutes. The aqueous phase was removed, water (1 mL) was added followed by trifluoroacetic acid (2 ml). The mixture was stirred at room temperature for 1 hour, water (10 ml) was added to the reaction mixture at 0° C. and stirred for 10 minutes. The organic phase was washed with saturated sodium hydrogen carbonate solution (2×15 ml) and water (2×15 ml), dried over magnesium sulfate and concentrated under reduced pressure. Purification via column chromatography (elutant: 50% to 100% dichloromethane in heptane) gave Compound 6 as a brown oil (15 mg, 62% yield).

### Step 5

Compound 6 (0.04 g, 0.029 mmol) was dissolved in chloroform (3 mL). The solution was degassed with nitrogen for 5 minutes and pyridine (0.02 mL, 0.29 mmol) was added. The solution was degassed for 15 minutes, cooled to 5° C. and Compound 7 (0.03, 0.112 mmol) was added as a solid in one portion. The mixture was allowed to warm to room temperature and stirred for 2 hours. Methanol was added and the mixture was concentrated to dryness. Purification by column chromatography (eluant: dichloromethane in heptane) gave Compound Example 1 (0.005 g. 9% yield).

### Compound Example 2

Compound Example 2 (NFA2) was prepared according to the following reaction scheme:

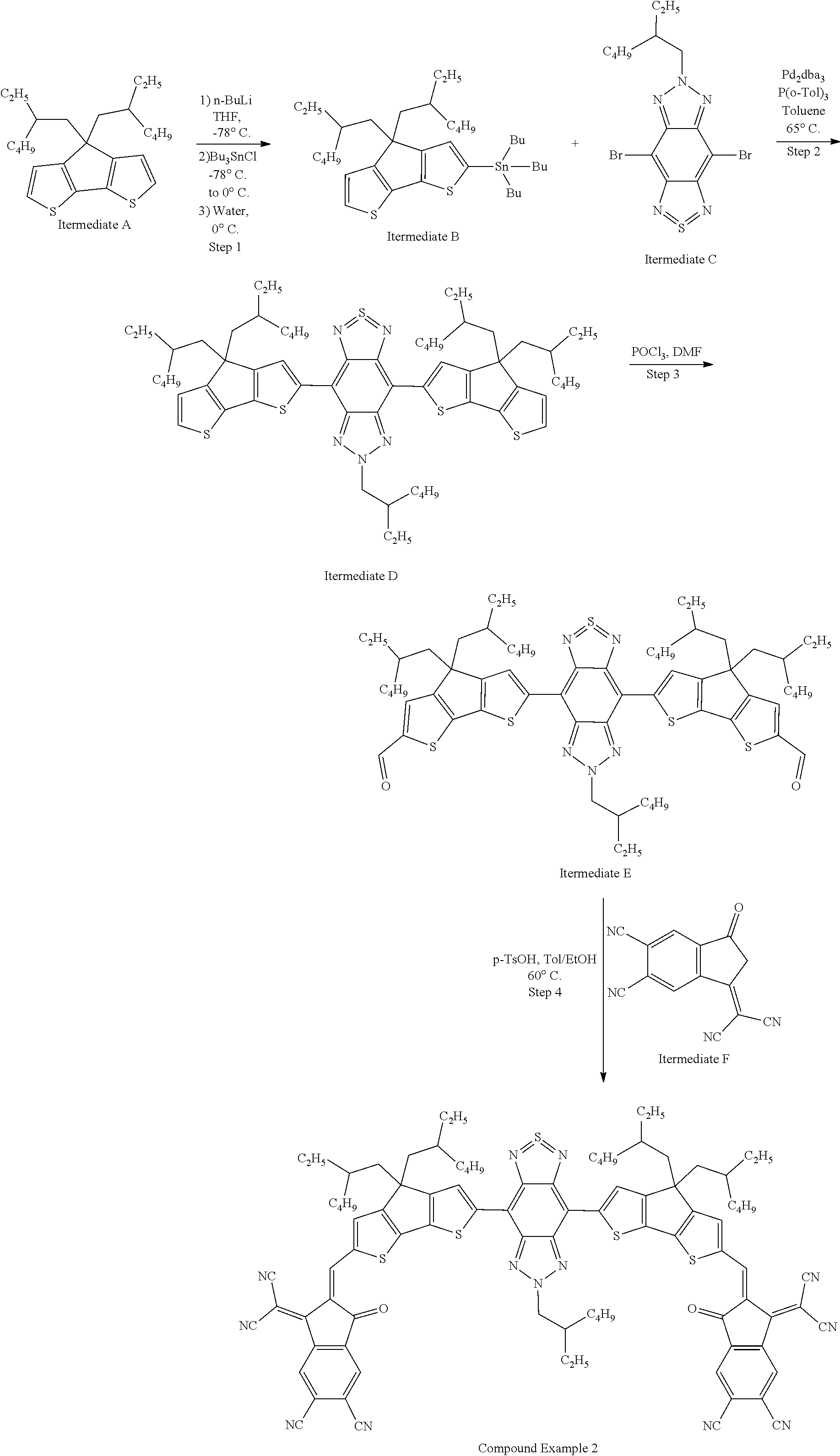

### Step 1

n-BuLi (1.01 mL, 2.53 mmol) was added to a solution of Intermediate A (1 g, 2.48 mmol) in THF at −78° C. under nitrogen and the mixture stirred for 3 hours. After this time tributyltin chloride (0.71 mL, 2.48 mmol) was added and the mixture was slowly warmed to room temperature over 3 hours. The mixture was cooled to 0° C., quenched with water, extracted with diethyl ether, washed with water and sat. NaCl, dried over MgSO₄ and filtered. The solvent was evaporated to give Intermediate A as a brown oil (1.65 g, 96% yield). The product was used in the next step without further purification. LCMS confirmed the mass of the expected product.

### Step 2

Tri(o-tolyl)-phosphine (0.09 g, 0.30 mmol) and Tris(dibenzylideneacetone)dipalladium(0) (0.07 g, 0.08 mmol) were added to a solution of Intermediate C (0.45 g, 0.99 mmol) and Intermediate B (1.63 g, 2.37 mmol) in toluene and the mixture was heated at 70° C. for 30 minutes, after which time the temperature was increased to 100° C. for a further 2 hours. The reaction mixture was then cooled, diluted with toluene and filtered through a silica plug and washed with toluene. Purification via column chromatography (eluant 5-10% of toluene in heptane) gave Intermediate D as deep green oil (1.03 g, 95.3% yield) LCMS confirmed the mass of the expected product.

### Step 3

Intermediate D (0.5 g, 0.46 mmol) was dissolved in DMF (15 ml) and cooled. Phosphorus oxychloride (0.43 mL, 4.58 mmol) was added dropwise at 2° C. The reaction mixture was stirred at this temperature for 1 hour and then at 80° C. for 2 hours after which time the reaction mixture was cooled to room temperature and saturated NaOAc was slowly. The mixture was diluted and extracted with DCM, organic phase was washed with saturated NaCl and water, dried over MgSO₄, filtered and evaporated. Purification via column chromatography (eluant: 16% to 100% dichloromethane:heptane) gave Intermediate E as a black oil (0.51 g, 97% yield). LCMS confirmed the mass of the expected product.

### Step 4

Intermediate E (0.51 g, 0.45 mmol), Intermediate F (0.57 g, 2.22 mmol) and p-TsOH (0.63 g, 3.33 mmol) is toluene (11.5 mL) and ethanol (23 mL) was heated to 65° C. under nitrogen. After 3 hours the reaction mixture was filtered, washed with methanol, ethanol, dichloromethane and pentane to give Compound Example 2 as a black solid (0.43 g 61% yield). LCMS confirmed the mass of the expected product.

### Compound Example 3

### Synthesis of Compound 1

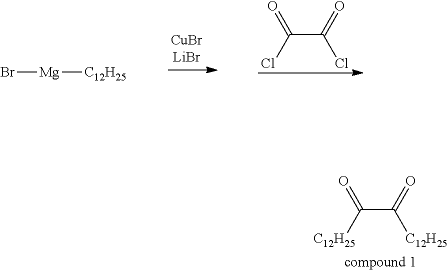

CuBr (5.24 g, 36.7 mmol) and LiBr (6.35 g, 73.1 mmol) was placed in a 500 ml round bottom flask containing a stirbar. The flask was flushed with dry N₂ gas and anhydrous THF (244 ml) was added. The reaction mixture was stirred for 15 min and then cooled to −78° C. in a dry ice/acetone bath. Then a 1.0M solution of C₁₂H₂₅MgBr in Et₂O (36.6 ml, 36.6 mmol) was added to the solution over 5 minutes. After 15 min, oxalyl chloride (2.23 g, 17.55 mmol) was added and then the reaction was stirred for 3 hours at −78° C. Then the reaction was allowed to warm to room temperature and stirred for 1 h. The reaction was quenched using a saturated aq. solution of ammonium chloride. The solvent was removed using rotary evaporation and the residue was treated with CHCl₃. The organic layer was washed with water and then dried over MgSO₄ and concentrated in vacuo to afford the crude product. After washing with hexane, recrystallization from CHCl₃ gave the compound 1 3.60 g (9.12 mmol, yield 52%) as a white solid. 1H-NMR (400 MHz, CHLOROFORM-D) δ2.71 (4H), 1.52-1.59 (4H), 1.24-1.27 (36H), 0.83-0.88 (6H)

### Synthesis of Compound 3

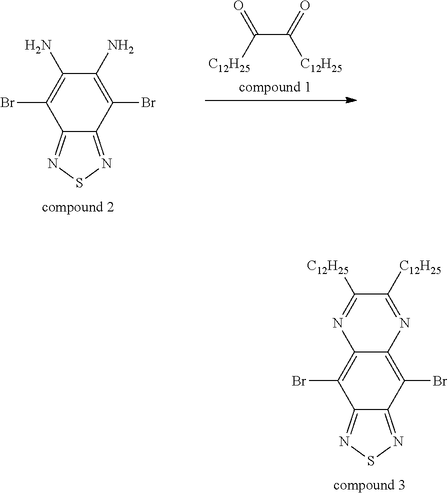

0.452 g (1.40 mmol) of compound 2, 0.551 g (1.40 mmol) of compound 1, 0.015 g of BHT (0.07 mmol) and 0.023 g of Mg₂SO₄ and were dissolved in 6.0 g of toluene and 6.0 g of AcOH and the mixture was stirred at 55° C. for 4 h. After cooling to room temperature, the mixture was poured into water and extracted with toluene. The organic extracts were washed with water twice and dried with anhydrous Na₂SO₄. The solvent was removed by vacuum distillation and the product was isolated by silica gel column chromatography (eluent: hexane/chloroform=50/50-10/90 wt %) to give compound 3 0.541 g (0.79 mmol, yield 57%). 1H-NMR (400 MHz, CHLOROFORM-D) δ3.08 (t, 4H), 1.94-2.02 (4H), 1.47-1.54 (4H), 1.16-1.45 (32H), 0.86 (6H)

### Synthesis of Compound 5

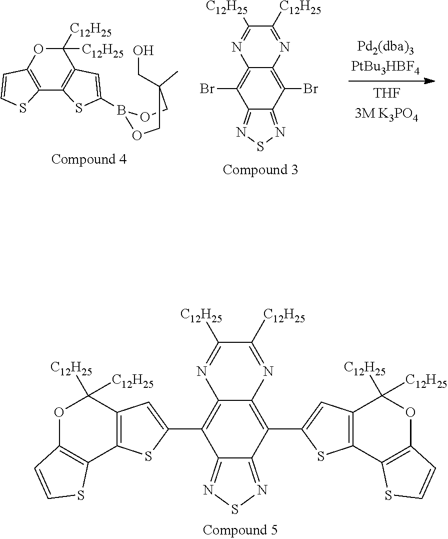

A mixture of compound 3 (0.53 g, 0.80 mmol), compound 4 (1.17 g, 1.77 mmol), Pd₂(dba)₃ (0.059 g, 0.06 mmol), P(tBu₃)HBF₄ (0.039 g, 0.13 mmol), THF (8.0 mL), and 3 M K₃PO₄ aq (8.0 mL) was heated at 60° C. for 2 hours under N₂. After cooling to room temperature, the organic layer was separated and after adding toluene, washed with water twice, dried over anhydrous MgSO₄ and filtered. After removing solvent, the resulting solid was purified by column chromatography on silica gel (hexane:chloroform 100:0-80:20 as eluent) to give compound 5 1.07 g (yield 84%) 1H-NMR (400 MHz, CHLOROFORM-D) δ 8.87 (s, 2H), 7.04 (d, 2H), 6.71 (d, 2H), 3.16 (t, J=7.5 Hz, 4H), 1.96-2.15 (12H), 1.17-1.54 (116H) 0.82-0.88 (18H)

### Synthesis of Compound 6

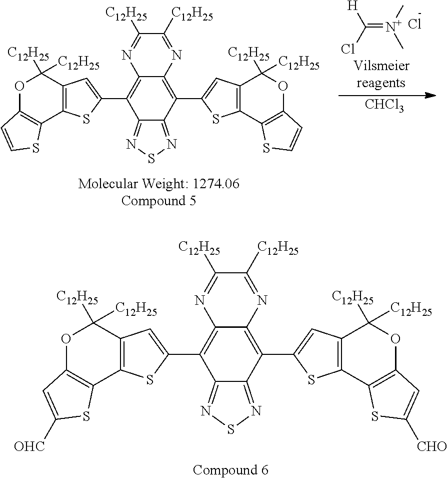

Compound 5 (1.00 g, 0.70 mmol) was dissolved in anhydrous chloroform (35 mL), followed by the addition of (chloromethylene)dimethyliminium chloride) (0.252 g, 1.97 mmol). This mixture was heated at 60° C. for 2 hours. After cooling to room temperature, saturated NaHCO₃ aq. was added and stirred for 10 minutes. The organic layer was separated and washed with water twice, dried over anhydrous MgSO₄ and filtered. After removing solvent, the resulting solid was purified by column chromatography on silica gel (hexane:ethyl acetate 96:4 as eluent) to give the compound 6 1.07 g (yield 93%). 1H-NMR (400 MHz, CHLOROFORM-D) δ 9.79 (s, 2H), 8.94 (s, 2H), 7.31 (s, 2H), 3.20 (t, J=7.5 Hz, 4H), 1.96-2.16 (m, 12H), 1.17-1.60 (m, 116H), 0.81-0.88 (m, 18H)

### Synthesis of Compound Example 3

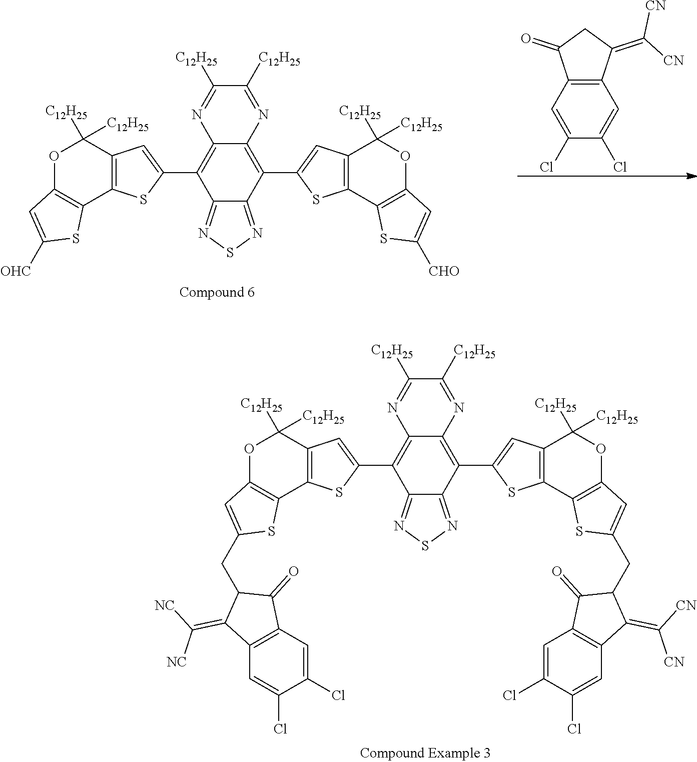

To a solution of compound 6 (450 mg, 0.275 mmol) and 2-(5,6-dichloro-3-oxo-indan-1-ylidene)-malononitrile (217 mg, 0.82 mmol) in anhydrous chloroform (13.6 g) was added pyridine (0.217 g, 2.74 mmol). The mixture was then degassed with nitrogen. The reaction mixture was warmed to 60° C. and stirred for 2 hours. The reaction mixture was then added to acetone and the resulting solid triturated with acetone with collection by filtration. The crude product was washed with mixture of toluene/hexane to give Compound Example 3 (277 mg, 47%) as a black solid.

¹H NMR (400 MHz, CDCl₃):δ 9.06 (s, 2H), 8.62 (2H), 8.56 (2H), 7.78 (2H), 7.40 (2H), 3.26 (t, 4H), 2.20-2.00 (m, 12H), 1.64 (m, 8H), 1.49-1.18 (m, 108H), 0.82 (m, 18H, —CH3).

### Compound Example 4

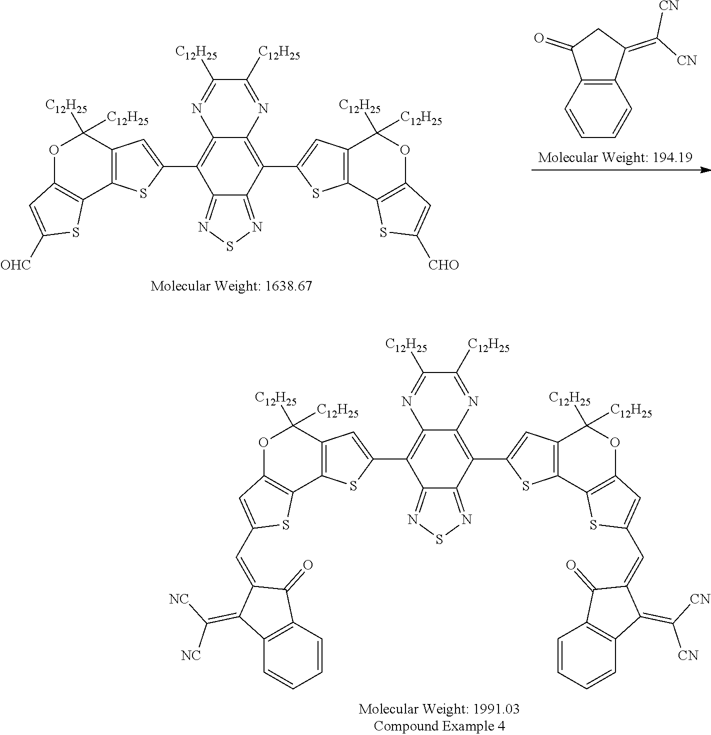

To a solution of compound 6 (630 mg, 0.384 mmol) and 2-(3-oxo-indan-1-ylidene)-malononitrile (224 mg, 1.15 mmol) in anhydrous chloroform (19 g) was added pyridine (0.304 g, 3.84 mmol). The mixture was then degassed with nitrogen. The reaction mixture was warmed to 60° C. and stirred for 3 hours. After cooling to room temperature, the precipitate was filtered and washed with chloroform and acetone to give Compound Example 4 (324 mg, 42%) as a black solid.

¹H NMR (400 MHz, CDCl₃):δ 9.04 (s, 2H), 8.68 (s, 2H), 8.60 (2H), 7.84 (2H), 7.69 (4H), 7.43 (s, 2H), 3.26 (t, 4H), 2.19-2.01 (m, 12H), 1.61 (m, 8H), 1.51-1.15 (m, 108H), 0.82 (m, 18H, —CH3).

### Compound Example 5

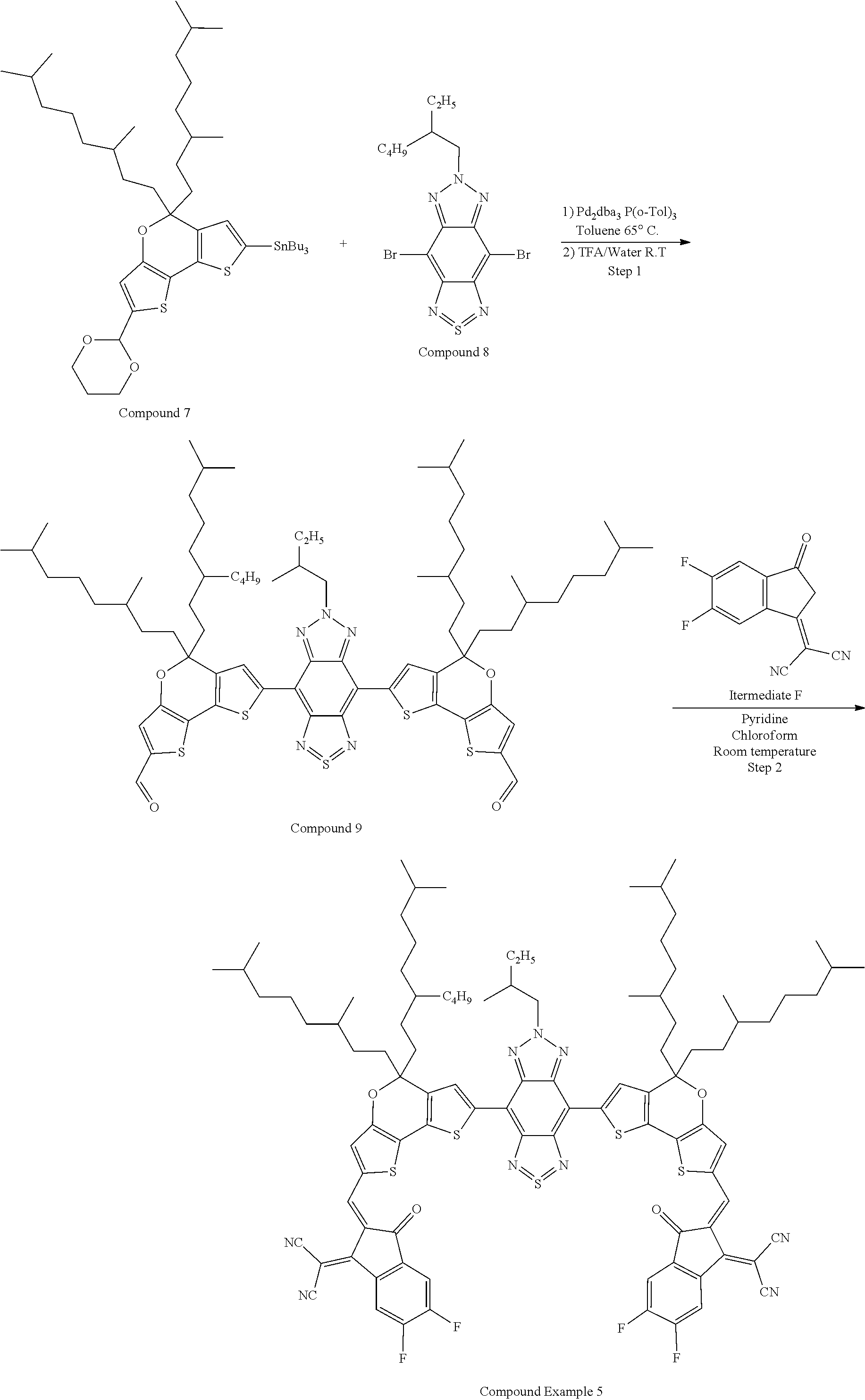

### Step 1

To degassed toluene (10 ml) solution, Compound 7 (1.175 g, 0.983 mmol) and Compound 8 (0.182 g, 0.409 mmol) were added. To this mixture catalyst-tris(dibenzylideneacetone) dipalladium (0.030 g, 0.030 mmol) followed by ligand-tris(o-tolyl)phosphine (0.030 g, 0.12 mmol) was added, the mixture was further degassed for 5 minutes and was then heated up to 70° C. for 2 hours. The reaction was cooled down to room temperature and 20 ml of water was added and the mixture was stirred for 5 minutes. The aqueous phase was removed, and 1 ml of water was added followed by trifluoroacetic acid (3 ml) added slowly. The mixture was stirred at room temperature for 3 hours. 20 ml of water was added to the reaction at 0° C. and stirred for 10 minutes. The phases were separated, the organic phase was washed with saturated sodium hydrogen carbonate solution followed by water, dried over magnesium sulfate and concentrated under reduced pressure. The crude product was purified via column chromatography with a dichloromethane/heptane mixture 60% to 100% of dichloromethane. After evaporation of fractions containing the product, Compound 9, 236 mg (45%) as a red oil was obtained. LCMS confirmed the expected mass of the product.

### Step 2

Compound 9 (0.263 g, 0.445 mmol), Intermediate F (0.246 g, 1.01 mmol) and p-TsOH (0.261 g, 1.52 mmol) were placed in a flask and toluene (6 mL) and ethanol (12 mL) were added. The mixture was heated up to 65° C. under nitrogen. After 3 hours methanol (20 mL) was added and the solution was filtered using a Buchner funnel, washed with methanol. The crude product was purified via column chromatography with chloroform. After evaporation of fractions containing the product was washed with heptane to give product, Compound Example 5, 0.113 g of a black solid (32%). LCMS confirmed the expected mass of the product.

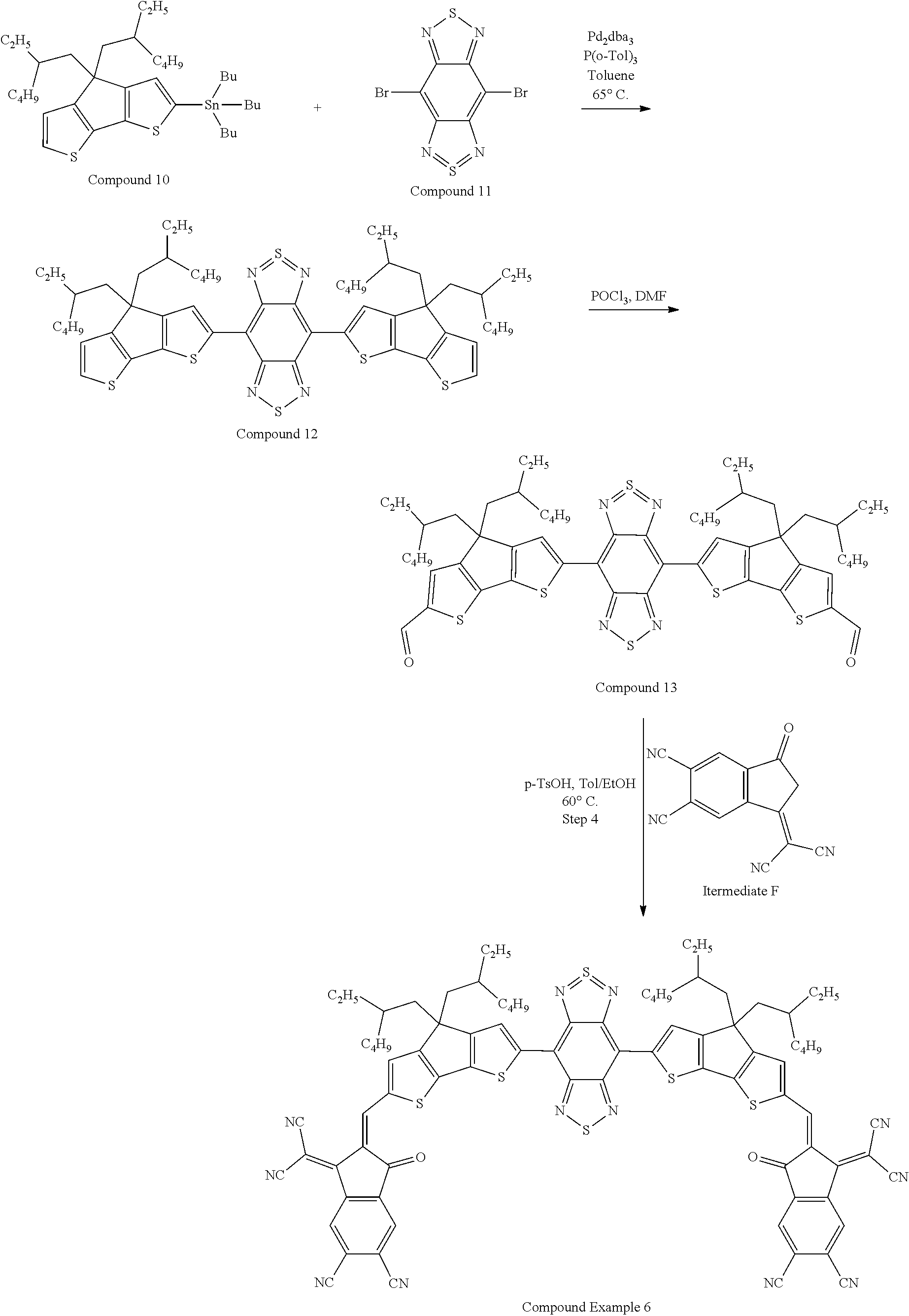

### Step 1

Compound 10 (1.64 g, 2.38 mmol) and Compound 11 (0.350 g, 0.994 mmol) were placed in a flask. Toluene (20 mL) was added and degassed for 15 minutes and then tri(o-tolyl)-phosphine (0.090 g, 0.298 mmol) and Tris(dibenzylideneacetone)dipalladium(0) (0.073, 0.079 mmol) were added. The mixture was further degassed for 5 minutes and heated at 70° C. for 30 minutes, after which the temperature was increased to 100° C. for a further stirred overnight. The reaction mixture was cooled down and solvent was removed on rotary evaporator. The crude product was purified via column chromatography with a heptane/toluene mixture. The solvent was evaporated to give Compound 12, 0.935 g (94.5%) of dark brown oil. LCMS confirmed the mass of the expected product.

### Step 2

Compound 12 (0.935 g, 0.939 mmol) was dissolved in DMF (30 ml) and cooled. Phosphorus oxychloride (0.87 mL, 9.39 mmol) was added dropwise at 3° C. The reaction mixture was stirred at this temperature for 1 hour and then at 80° C. for 2 hours after which the reaction mixture was cooled to room temperature and saturated NaOAc was added to it slowly. The mixture was diluted and extracted with DCM. The organic phase was washed with saturated NaCl and water, dried over MgSO₄, filtered and evaporated. The product was purified via column chromatography with a dichloromethane:heptane mixture of 16% to 100% dichloromethane. Fractions with the product were evaporated to give Compound 13, 0.665 g (67%) as dark red sparkly foamy solid. LCMS confirmed the mass of the expected product.

### Step 3

Compound 13 (0.660 g, 0.628 mmol), Intermediate F (0.800 g, 3.130 mmol) and p-TsOH (0.893 g, 4.700 mmol) were placed in a flask and toluene (16 mL) and ethanol (32 mL) were added. The mixture was heated up to 65° C. under nitrogen. After 4 hours the reaction mixture was filtered using a Buchner funnel, washed with methanol and ethanol. Crude product was purified via column chromatography with dichloromethane:heptane solvent mixture 50% to 100% of dichloromethane. Fractions containing product were evaporated and suspended in dichloromethane:chloroform mixture and filtrated, washed with toluene and heptane to give Compound Example 6, 0.496 g (52%) as a black solid, LCMS confirmed the expected mass of the product.

### Compound Example 7

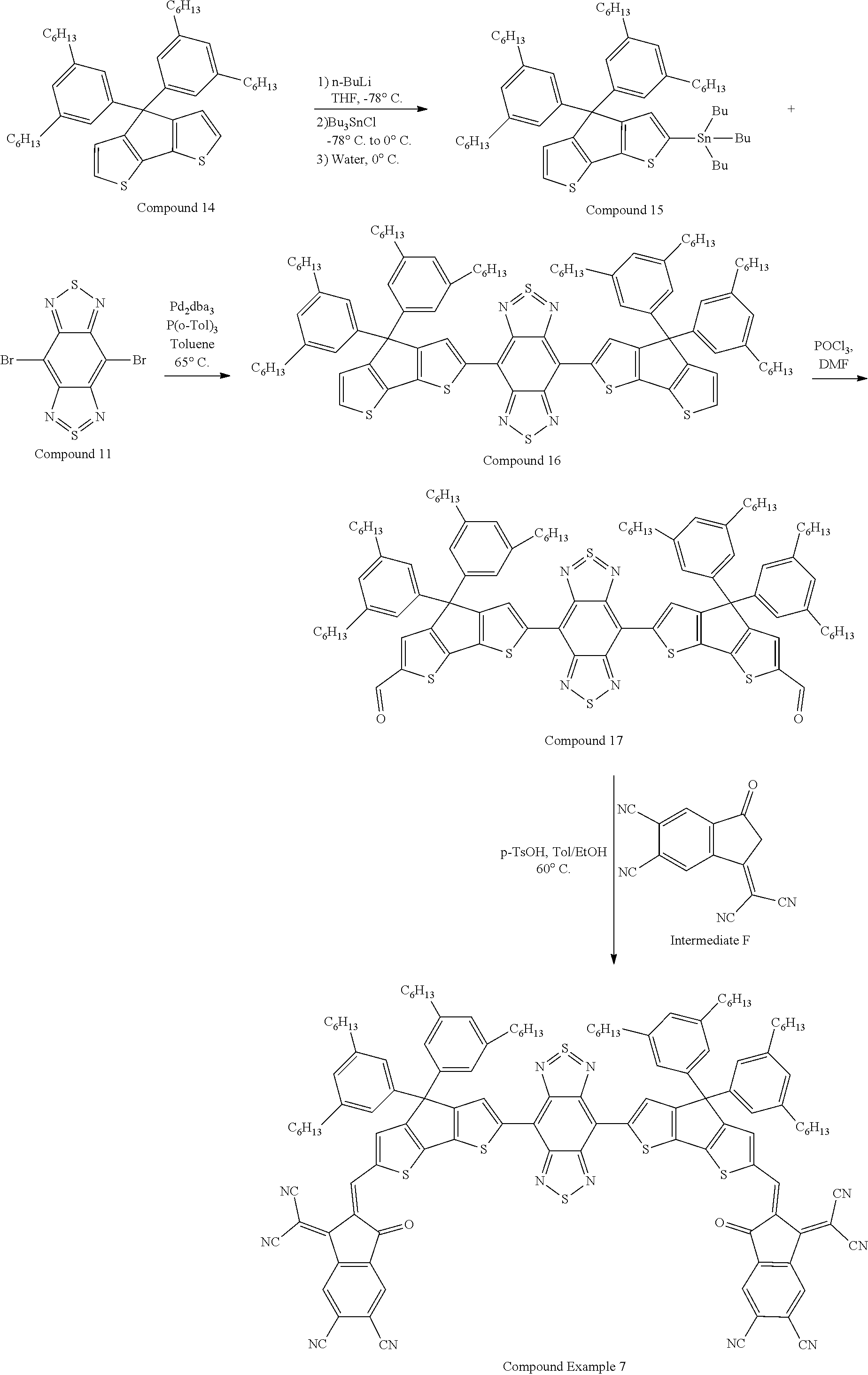

### Step 1

Compound 14 (4.2 g, 6.25 mmol) was placed in a flask under N₂, dissolved in THE (55 mL) and cooled to −78° C. n-BuLi (2.55 mL, 6.37 mmol) was added and the mixture was stirred at this temperature for 3 hours. After this time warmed up to −70° C., tributyltin chloride (1.79 mL, 6.25 mmol) was added and the mixture was brought −15° C. The mixture slowly warmed up to room temperature and stirred overnight. The mixture was brought to 0° C., quenched with water, extracted with diethyl ether, washed with water and sat. NaCl, dried over MgSO₄ and filtered. Most of the solvent was evaporated to give Compound 15, 6.545 g (109%) as a brown/orange oil. LCMS confirmed the expected product, used in the next step without further purification.

### Step 2

Compound 15 (6.545 g, 5.12 mmol) and Compound 11 (0.837 g, 2.38 mmol) were placed in a flask. Toluene (43 mL) was added and degassed for 15 minutes and then tri(o-tolyl)-phosphine (0.217 g, 0.714 mmol) and tris(dibenzylideneacetone)dipalladium(0) (0.174, 0.190 mmol) were added. The mixture was further degassed for 5 minutes and heated at 70° C. for 1 hour, after which the temperature was increased to 100° C. for a further stirred overnight. The reaction mixture was cooled down and solvent was removed on rotary evaporator. The crude product was purified via column chromatography with heptane/toluene mixture, 0% to 20% of toluene. Fractions with product were evaporated to give Compound 16, 3.212 g (89%) as a dark brown oil. LCMS confirmed the mass of the expected product.

### Step 3

Compound 16 (3.212 g, 2.10 mmol) was dissolved in toluene (12 mL) and heptane mixture (20 mL). To this DMF (70 ml) was added and the mixture was cooled down with ice bath. Phosphorus oxychloride (1.95 mL, 21.0 mmol) was added dropwise at 3° C. The reaction mixture was stirred at this temperature for 1 hour and then at 80° C. for 2 hours after which the reaction mixture was cooled to room temperature and saturated sodium acetate was added to it slowly. The mixture was diluted and extracted with toluene. The organic phase was washed with saturated NaCl and water, dried over MgSO₄, filtered and evaporated. The product was purified via column chromatography with a dichloromethane:heptane mixture of 16% to 100% dichloromethane. Fractions with the product were evaporated to give Compound 17, 2.719 g (82%) as deep red sparkly foamy solid. LCMS confirmed the mass of the expected product.

### Step 4

Compound 17 (1.700 g, 1.07 mmol), Intermediate F (1.36 g, 5.35 mmol) and p-TsOH (1.52 g, 4.700 mmol) were placed in a flask and toluene (27 mL) and ethanol (55 mL) were added. The mixture was heated up to 65° C. under nitrogen. After ˜4 hours the reaction mixture was filtered using a Buchner funnel, washed with methanol and ethanol. Crude product was purified via column chromatography with chloroform:heptane solvent mixture from 50% to 100% of chloroform. Fractions containing product were evaporated and dissolved in dichloromethane and precipitated into pentane, washed with dichloromethane and pentane to give Compound Example 7, 0.298 g (21%) as a black solid, LCMS confirmed the expected mass of the product.

### Compound Example 8

### Synthesis of Compound 18

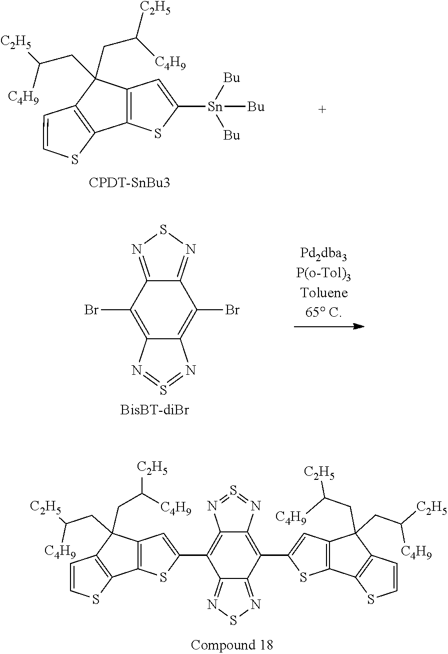

Toluene (60 ml) was added to CPDT-SnBu₃ (4.84 g, 7.00 mmol) and BisBT-diBr (1.15 g, 3.26 mmol) under nitrogen. The mixture was degassed for 15 minutes and tris(2-methylphenyl)phosphine (0.30 g, 0.98 mmol) and tris(dibenzylideneacetone) dipalladium (0.24 g, 0.26 mmol) were added and the mixture was degassed for additional 5 minutes. The mixture was heated at 70° C. for 30 minutes and then at 100° C. overnight. Upon completion, solvent was removed on a rotary evaporator and purification by column chromatography (silica gel; heptane/toluene) gave Compound 18 (2.37 g) as a dark brown oil.

### Synthesis of Compound 19

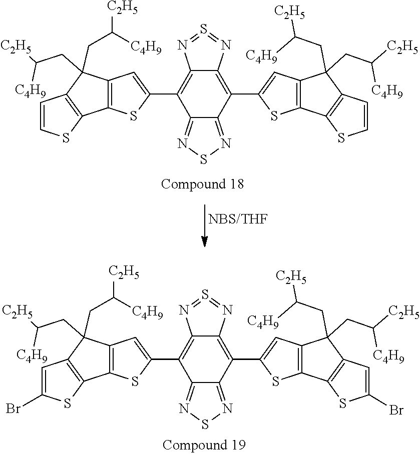

A solution of Compound 18 (0.5 g, 0.50 mmol) in THF (5 ml) and cooled to −40° C. and N-bromosuccinimide (0.18 g, 1 mmol) was added portion-wise. The mixture was stirred at this temperature for 4.5 hours and quenched with 10% sodium thiosulfate solution, extracted with heptane, dried over magnesium sulphate, and evaporated to give Compound 19 as a black oil.

### Synthesis of Compound 20

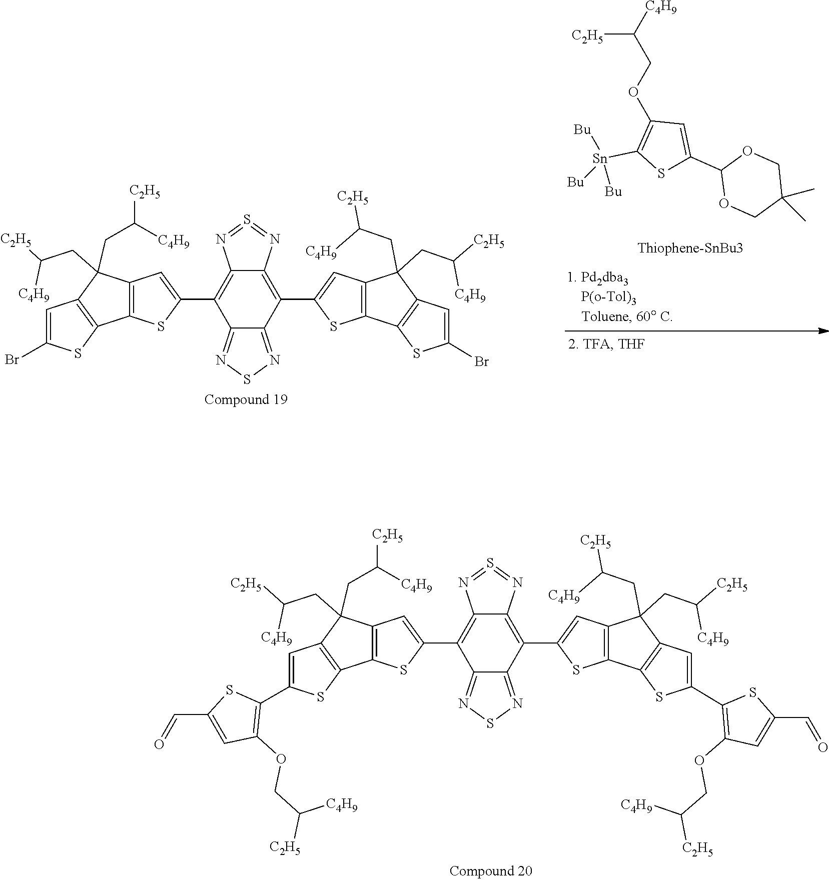

A solution of Compound 19 (0.53 g, 0.46 mmol) and Thiophene-SnBu₃ (1.03 g, 1.67 mmol) in toluene (9 ml) was degassed for 15 minutes. Tris(2-methylphenyl)phosphine (0.04 g, 0.14 mmol) and tris(dibenzylideneacetone) dipalladium (0.03 g, 0.04 mmol) were added and the mixture was degassed for additional 5 minutes. The mixture was heated at 70° C. for 30 min and then at 100° C. overnight. Upon completion it was diluted with toluene and extracted with water. The organic phase was placed in a flask, trifluoroacetic acid (4 ml) was added, and it was stirred for 30 minutes at room temperature and then at 40° C. for another 30 minutes. The reaction mixture was cooled to room temperature, water (10 ml) was added followed by a saturated solution of sodium hydrogen carbonate, it was transferred to separating funnel and further extracted with this solution. The organic phase was dried over magnesium sulphate, filtered and concentrated under vacuum to give a purple oil. Purification via column chromatography (silca-gel; heptane/toluene) gave Compound 20 (0.25 g) as a purple solid.

### Synthesis of Compound Example 8

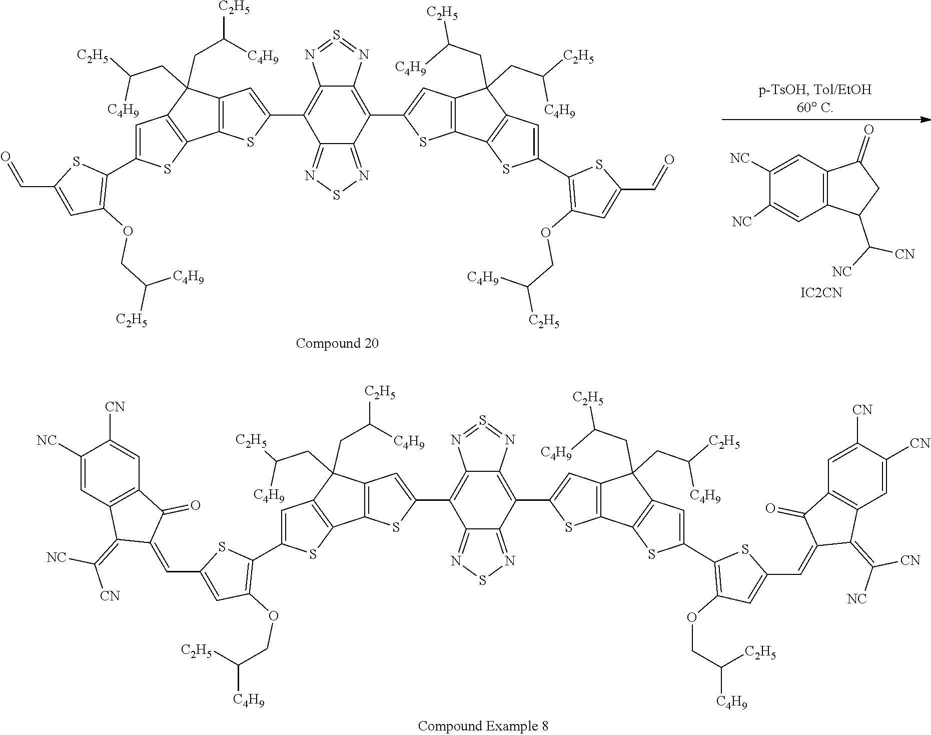

Compound 20 (0.25 g, 0.17 mmol), IC2CN (0.21 g, 0.83 mmol) and para-toluenesulfonic acid (0.24 g, 1.24 mmol) were placed in a flask, toluene (6 ml) and ethanol (8 ml) were added, and the mixture was degassed for 15 minutes with nitrogen and heated at 70° C. overnight. After this time the mixture was filtered and the resulting solid was washed with hot ethanol, methanol and pentane. Purification via column chromatography (silica gel; toluene DCM and THF) gave Compound Example 8 (0.07 g).

¹H NMR (300 MHz, THF-d₈): δ 9.28 (s, 2H), 8.98 (s, 2H), 8.79 (s, 2H), 8.35 (s, 2H), 7.97 (t, 2H), 7.83 (s, 2H), 4.35 (d, 4.7 Hz, 4H), 2.22 (m, 8H), 1.96 (m, 2H), 1.49-1.44 (m, 9H), 1.12-0.93 (m, 54H), 0.75-0.60 (m, 27H).

LCMS (APCI+ve): 1924.91 ([M+H]+).

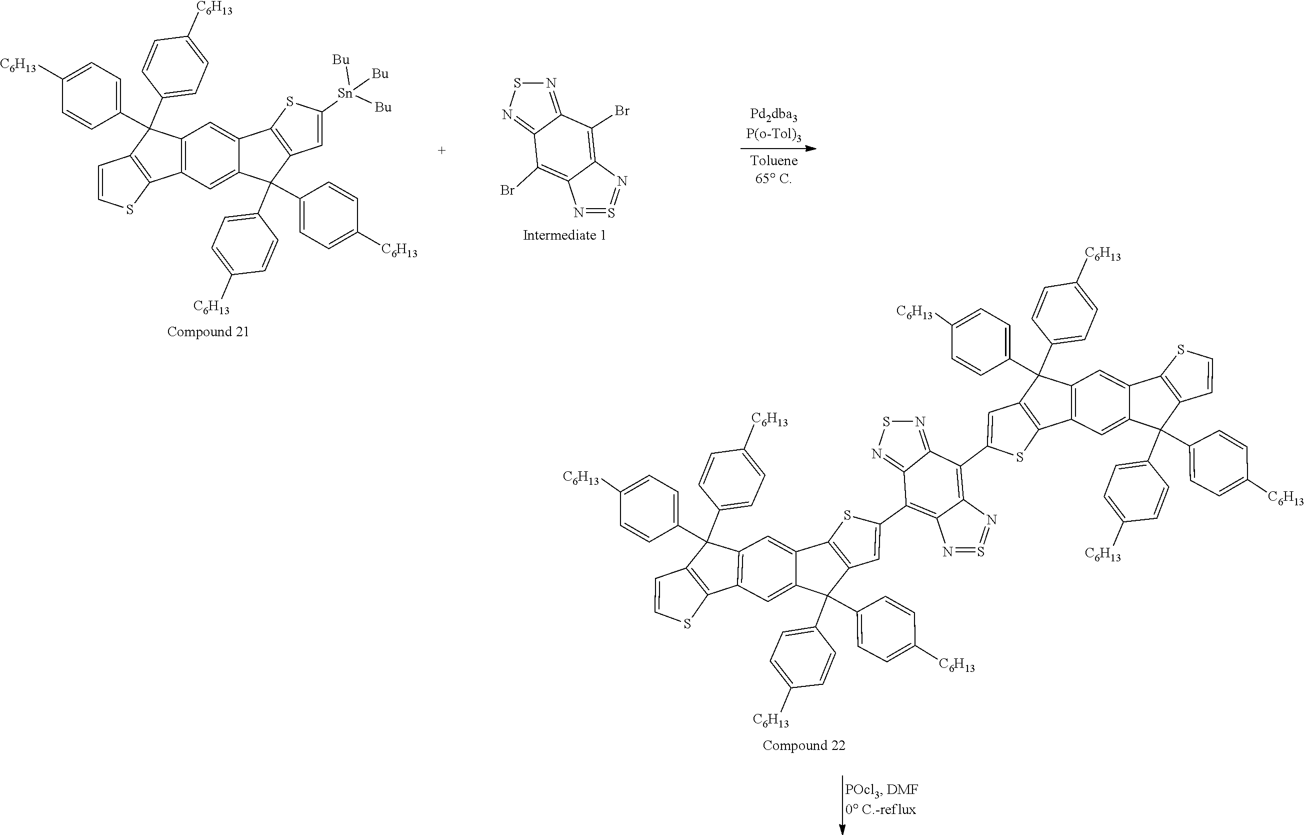

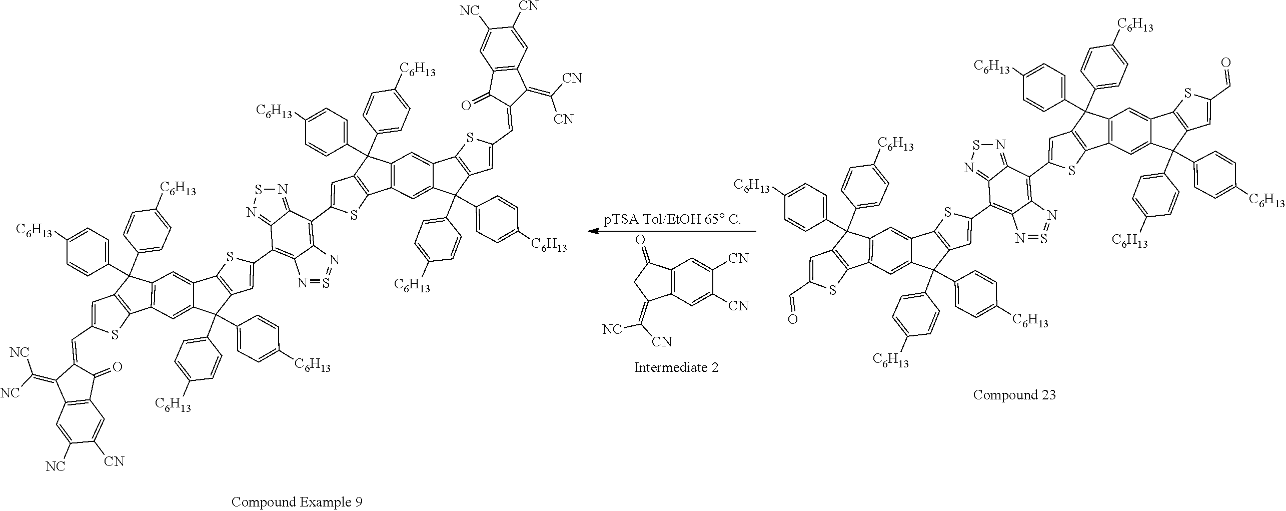

Compound 21 was prepared as described in Zhang et al, “Electron-Deficient and Quinoid Central Unit Engineering for Unfused Ring-Based A₁-D-A₂-D-A₁-Type Acceptor Enables High Performance Nonfullerene Polymer Solar Cells with High V_{oc} and PCE Simultaneously” Small 2020, 16, 1907681.

### Compound 22

A solution of Compound 21 (3.94 g, 1.97 mmol) in toluene (100 mL) was degassed for 45 minutes. Intermediate 1 (0.29 g, 0.82 mmol), Pd₂(dba)₃ (0.06 g, 0.07 mmol), and P(o-Tol)₃ (0.08 g, 0.25 mmol) were added, and the mixture was heated to 65° C. for 3.5 hours under nitrogen and stirred at room temperature overnight. After this time, additional catalyst (60 mg) and ligand (75 mg) were added, and the solution was heated to 100° C. for 1 hour. An additional 90 mg of BisBT-diBr was added (50 mg), and the solution was heated at 100° C. for a further 4.5 hours. After this time the mixture was cooled to room temperature and the solvent was removed in vacuo to give a red/brown solid. The crude residue was purified by column chromatography (heptane/dichloromethane and toluene/heptane. to give Compound 22 (100 g) as a dark red/brown solid.

### Compound 23

Compound 22 (0.90 g, 0.45 mmol) was added to DMF (30 mL) under nitrogen and the mixture was heated gently until it dissolved. The mixture was then cooled to −2° C. with an ice bath. POCl₃ (0.45 mL, 4.47 mmol) was added dropwise (maintaining temperature <5° C.). Once the addition was complete the mixture was stirred at room temperature for 15 minutes before being heated to 80° C. for 3.5 hours. The reaction was then cooled to room temperature and quenched with saturated sodium acetate, then additional water was added. The mixture was extracted three times with toluene and the organic phases were combined and extracted with aqueous NaCl to remove any residual DMF. The crude residue was purified by column chromatography (toluene) to give Compound 23 (0.99 g) as a red/brown solid.

### Compound Example 9

Compound 23 (0.63 g, 0.30 mmol), Intermediate 2 (0.39 g, 1.52 mmol) and p-toluenesulfonic acid (0.43 g, 2.28 mmol) were placed in a flask under nitrogen. Toluene (8 mL) and ethanol (16 mL) were added and the mixture purged with nitrogen for 15 minutes. It was then was heated up to 65° C. overnight. After cooling to room temperature, it was filtered, washed twice with hot EtOH (25 mL), three times with hot MeOH (25 mL), and three times with pentane (25 mL) to give 0.66 g of a black solid The crude solid was purified via column chromatography (dichloromethane/heptane), dissolved in dichloromethane (10 mL) and reprecipitated into pentane (100 mL) to give Compound Example 9 (0.23 g) as a black solid.

¹H NMR (300 MHz, THF-d₈): δ 9.15 (bs, 2H), 9.01 (d, 3.93 Hz, 2H), 8.42 (s, 2H), 8.12 (s, 2H), 8.05 (bs, 2H), 7.88 (s, 2H), 7.38 (d, 8.11 Hz, 8H), 7.27 (d, 7.73 Hz, 8H), 7.13 (t, 7.93 Hz, 16H), 2.58 (t, 7.01 Hz, 16H), 1.62-1.57 (m, 16H), 1.36-1.28 (m, 48H), 0.89-0.85 (m, 24H).

LCMS (APCI+ve): 2513.45 ([M]+).

### Compound Example 10

A compound in which the donor groups are different may be prepared according to the following reaction scheme.

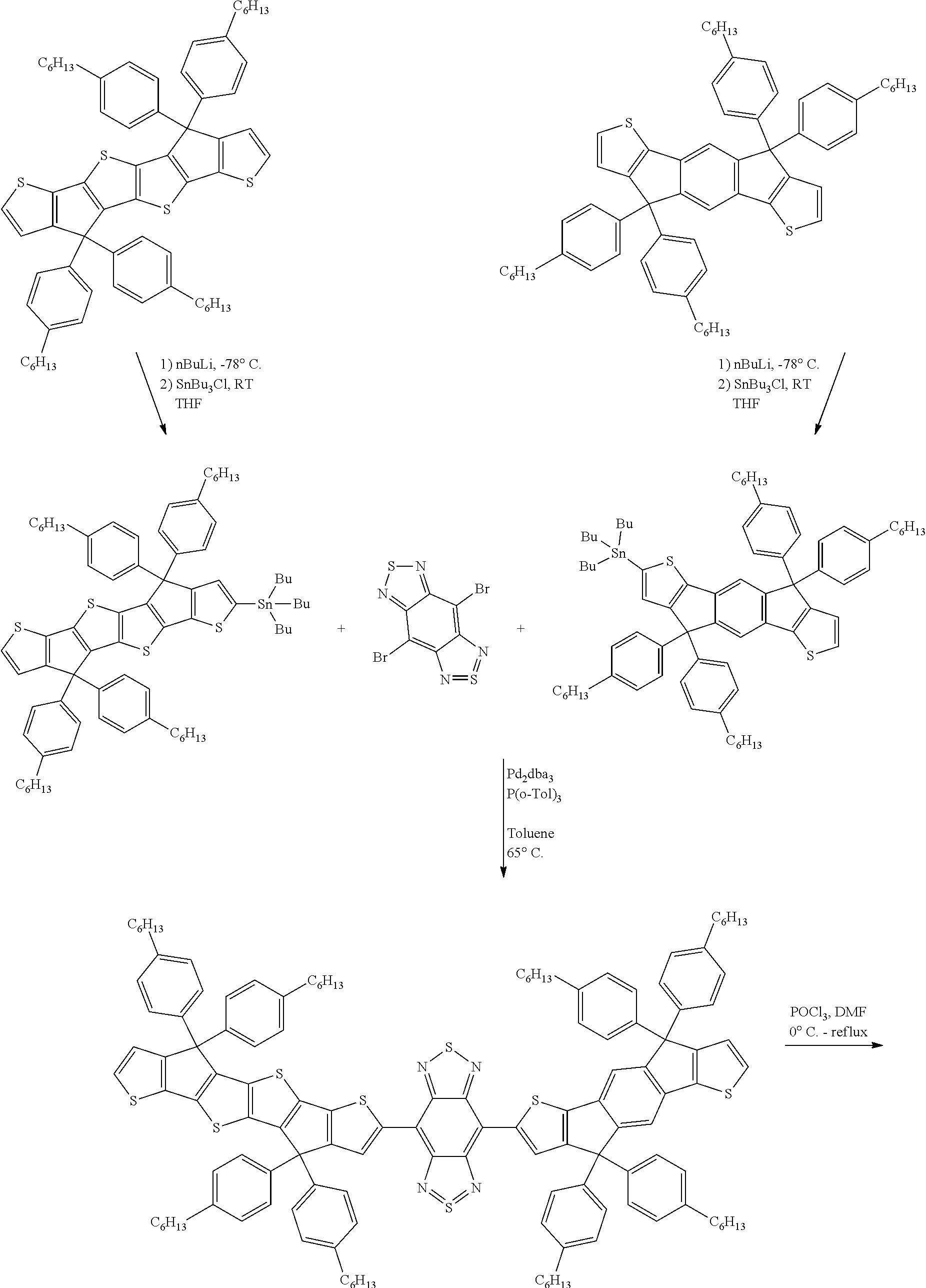

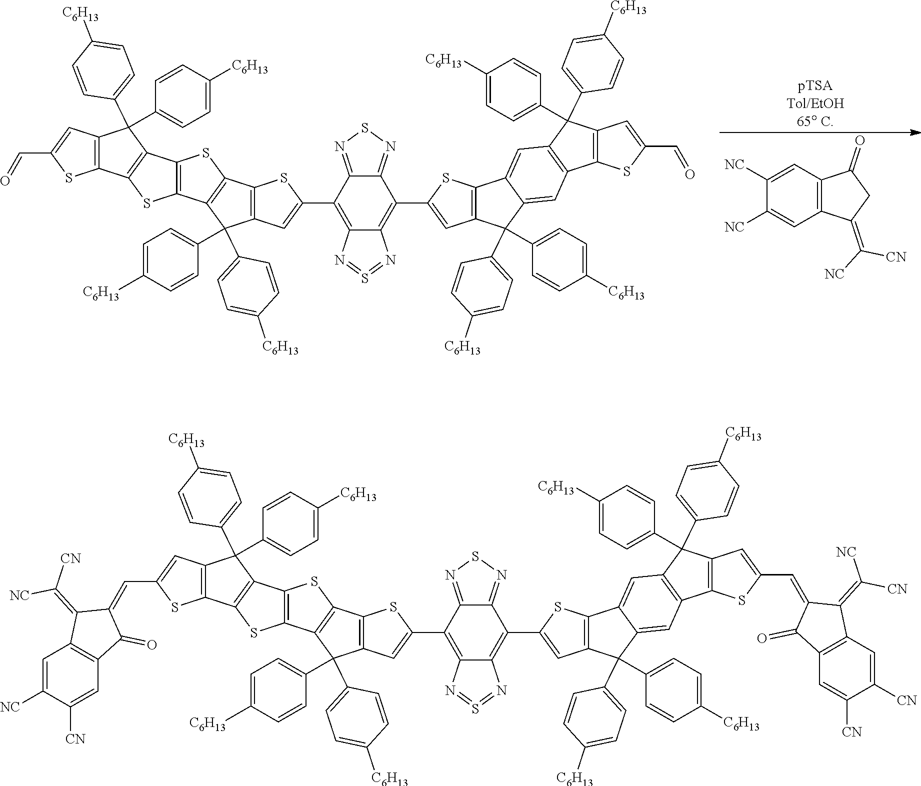

### Absorption Measurements

Absorption spectra for Compound Examples 1-4 were measured in xylene solution or in a film formed by spin-coating a xylene solution of the compound.

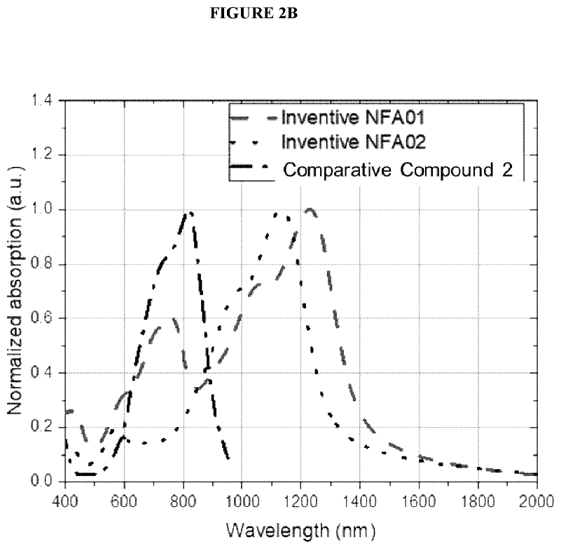

FIG. 2A shows the absorption spectrum of films of Compound Examples 1 and 2 compared to the Comparative Compound 1:

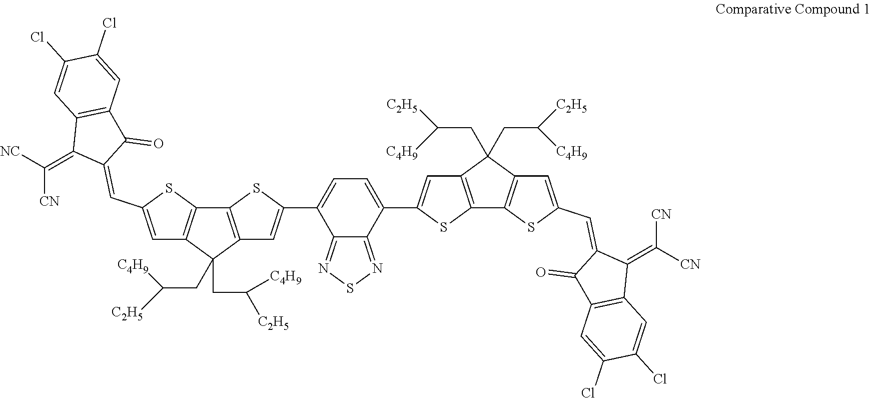

The absorption spectrum for Comparative Compound 1 is taken from Pang et al, “Nonfused Nonfullerene Acceptors with an A-D-A′-D-A Framework and a Benzothiadiazole Core for High-Performance Organic Solar Cells”, ACS Appl. Mater. Interfaces 2020, 12, 14, 16531-16540.

FIG. 2B shows the absorption spectrum of films of Compound Examples 1 and 2 compared to the Comparative Compound 2:

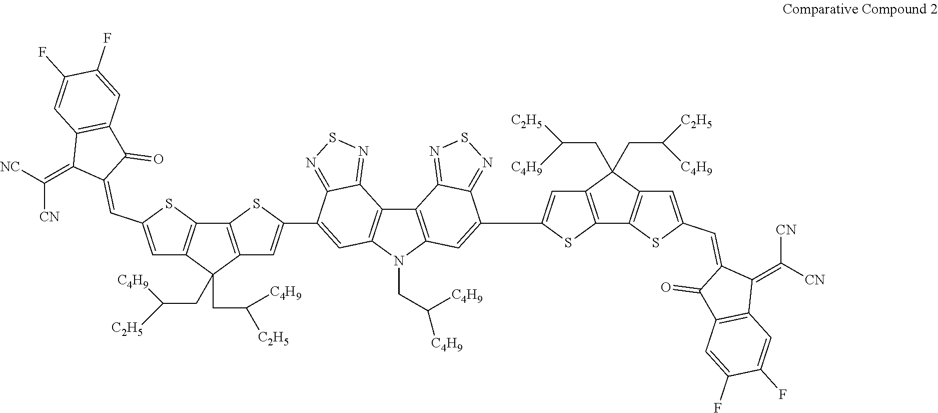

The absorption spectrum for Comparative Compound 2 is taken from CN11260833.

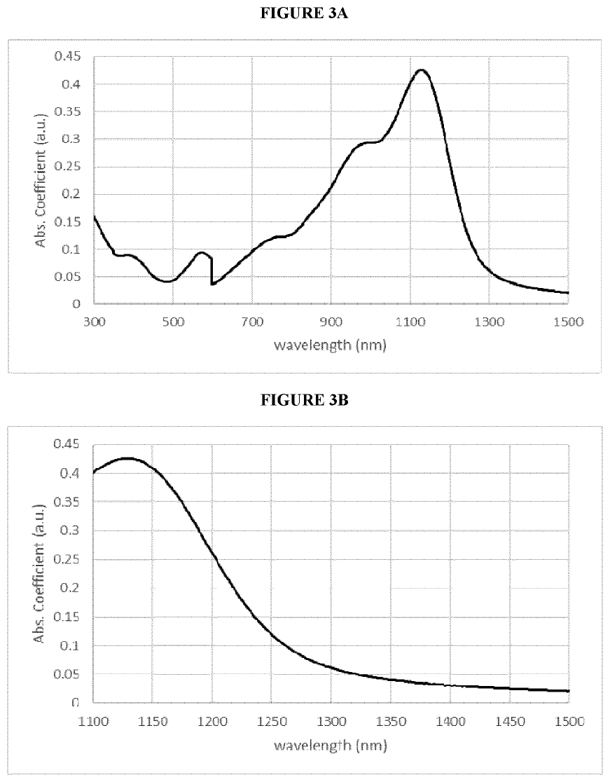

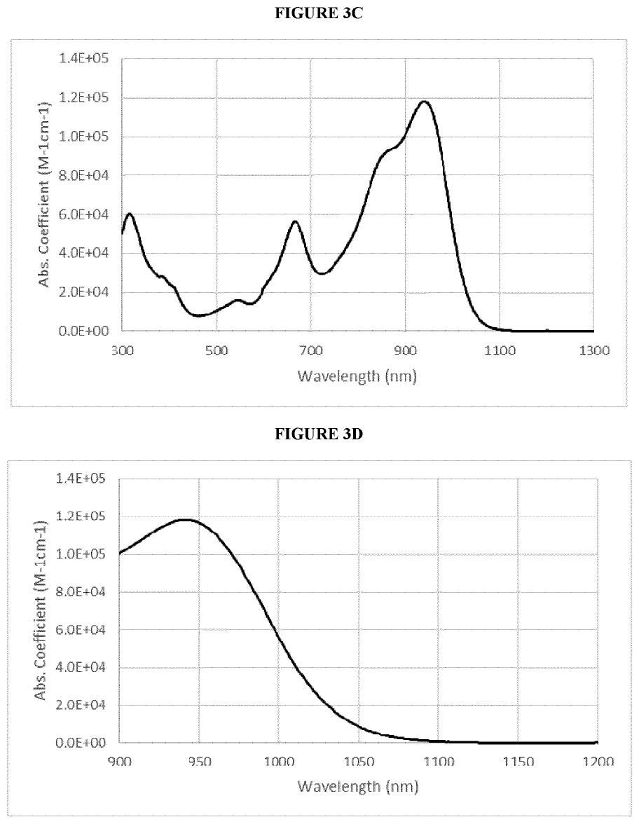

FIGS. 3A and 3B show the film absorption spectrum of Compound Example 3.

FIGS. 3C and 3D show the solution absorption spectrum of Compound Example 3.

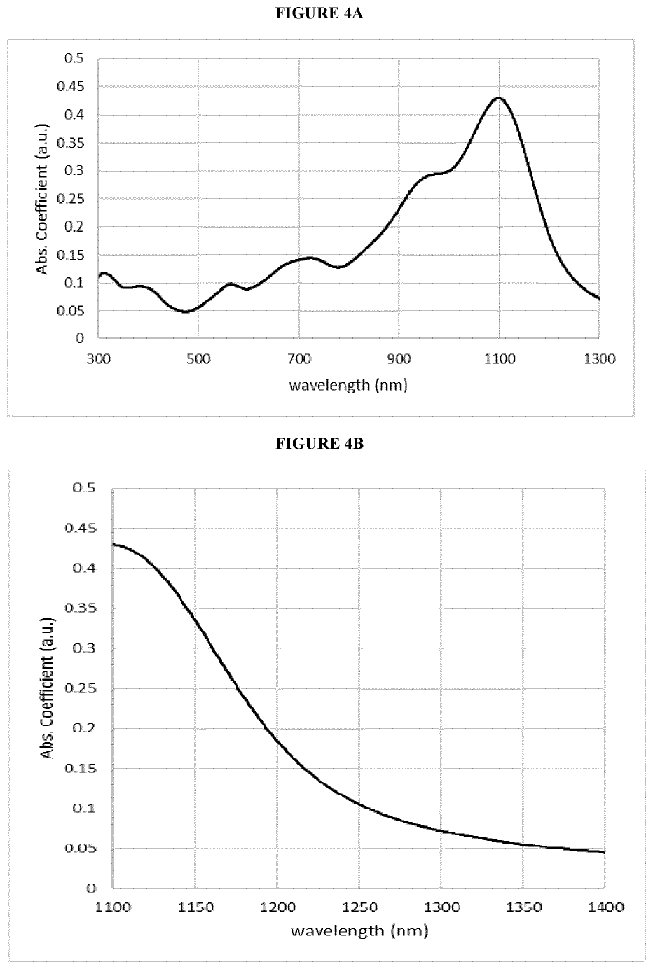

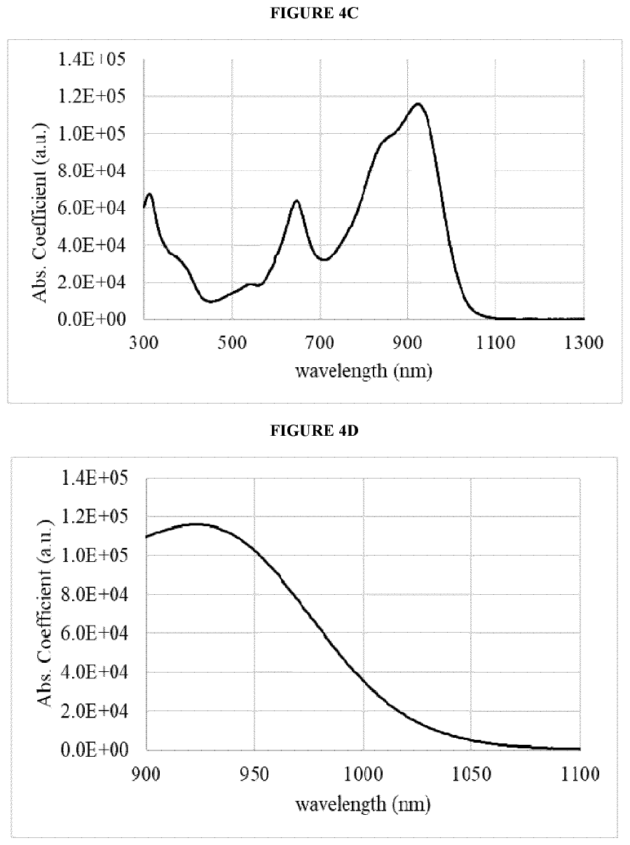

FIGS. 4A and 4B show the film absorption spectrum of Compound Example 4.

FIGS. 4C and 4D show the solution absorption spectrum of Compound Example 4.

Each of Compound Examples 1-4 show strong absorption in film at wavelengths above 1000 nm.

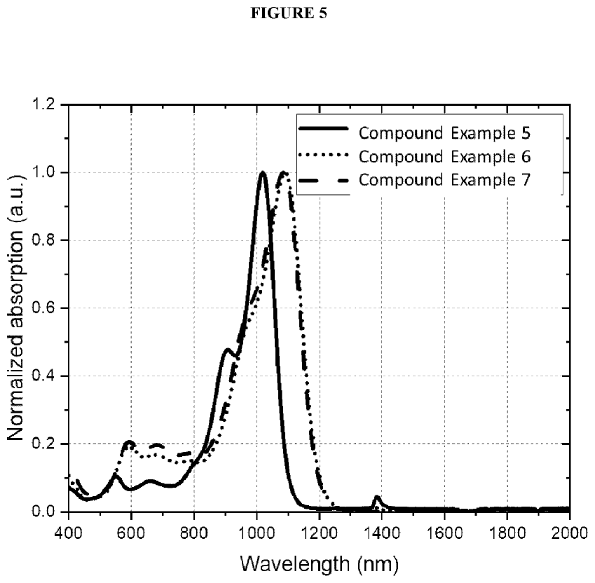

Absorption spectra for Compounds 5-7 in 1×10⁻⁶ M o-dichlorobenzene solution are shown in FIG. 5.

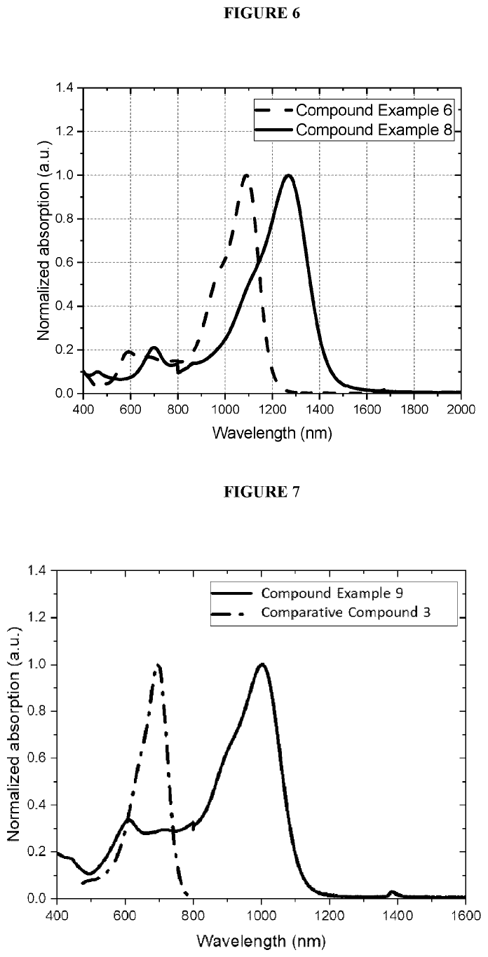

An absorption spectrum for an o-dichlorobenzene solution of Compound Example 8 containing thiophene bridges is shown in FIG. 6. For comparison, the corresponding o-dichlorobenzene solution spectrum for unbridged analogue Compound Example 6 is also shown. The presence of the bridges of Compound Example 8 results in a shift to a longer absorption wavelength.

An absorption spectrum for Compound Example 9 in o-dichlorobenzene solution is shown in FIG. 7. This is overlaid with the absorption spectrum of Comparative Compound 3, illustrated below, which is reproduced from Zhang et al, “Electron-Deficient and Quinoid Central Unit Engineering for Unfused Ring-Based A₁-D-A₂-D-A₁-Type Acceptor Enables High Performance Nonfullerene Polymer Solar Cells with High V_{c} and PCE Simultaneously”, Small, DOI: 10.1002/smll.201907681.

As shown in FIG. 7, the absorption peak of Compound Example 9 is at around 1000 nm whereas that of Comparative Compound 3 is around 700 nm.

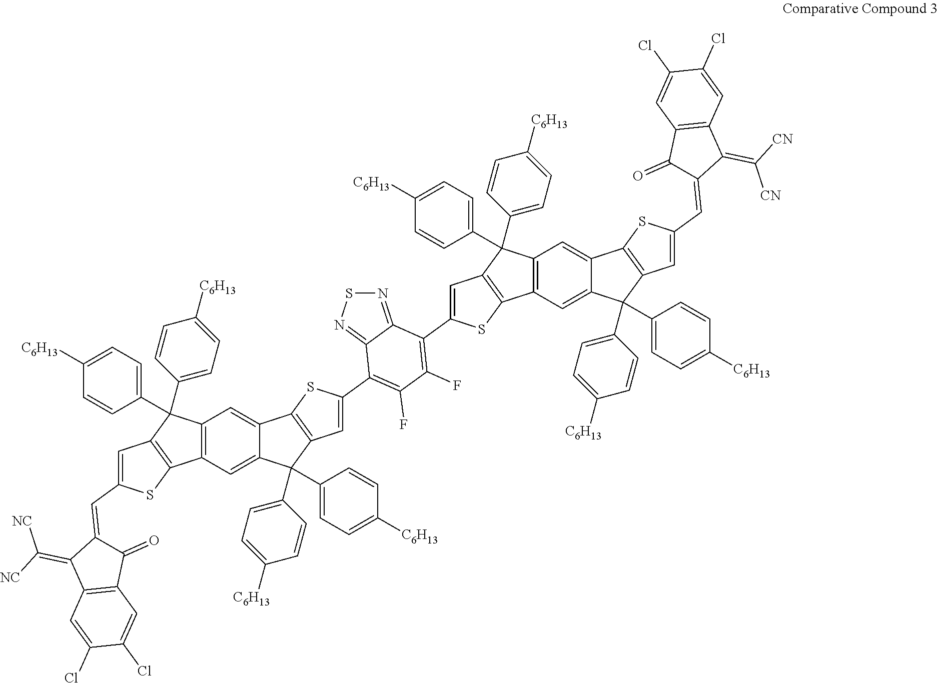

### Square Wave Voltammetry Measurements

HOMO and LUMO energy levels of films of the example compounds were measured by square wave voltammetry.

| Compound | HOMO/eV | LUMO/eV |
|---|---|---|
| Compound example 1 | −5.54 | −3.92 |
| Compound example 2 | −5.40 | −4.10 |
| Compound Example 5 | −5.32 | −4.22 |
| Compound Example 6 | −5.41 | −4.15 |
| Compound Example 7 | −5.49 | −4.19 |

### Device Example 1

An organic photodetector having the following structure was prepared:

Cathode/Donor:Acceptor layer/Anode

A glass substrate coated with a layer of indium-tin oxide (ITO) was treated with polyethyleneimine (PEIE) to modify the work function of the ITO.

A mixture of Donor Polymer 1 (donor) and Compound Example 1 (acceptor) in a donor acceptor mas ratio of 1:1.5 was deposited over the modified ITO layer by blade coating from a 15 mg/ml solution in 1,2,4 Trimethylbenzene; 1,2-Dimethoxybenzene 95:5 v/v solvent mixture. The film was dried at 80° C. to form a ca. 500 nm thick bulk heterojunction layer.

Donor Polymer 1 is a donor-acceptor polymer shown below, having a donor repeat unit of formula (VIIa) and an acceptor repeat unit. Donor Polymer 1 may be prepared as described in WO2013/051676, the contents of which are incorporated herein by reference.

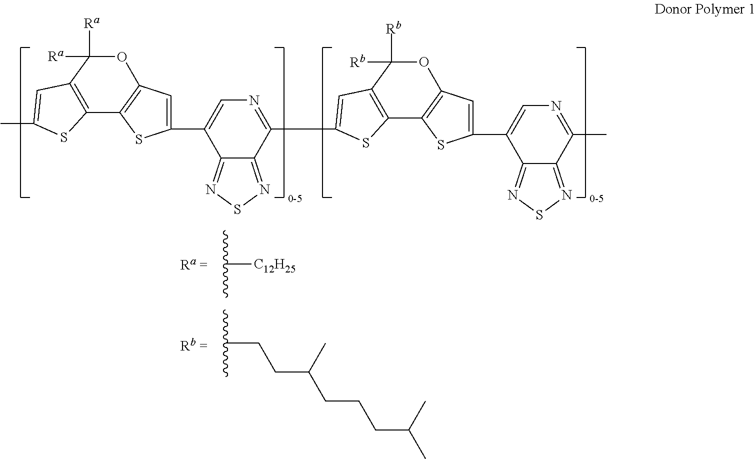

Donor Polymer 1 has a peak absorption wavelength of 933 nm.

An anode stack of MoO₃ (10 nm) and ITO (50 nm) was formed over the bulk heterojunction by thermal evaporation (MoO₃) and sputtering (ITO).

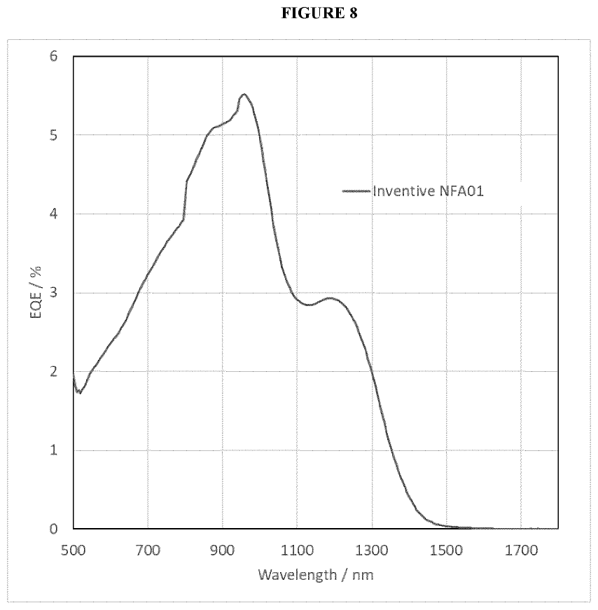

External quantum efficiency (EQE) was measured across a range of wavelengths. Results are shown in FIG. 8.

### Device Example 2

An organic photodetector was prepared as follows:

A formulation of Donor Polymer 2 (1 wt %) and Compound Example 3 (1 wt %) in ortho-dichlorobenzene was deposited by spin-coating onto a glass substrate coated with a layer of indium-tin oxide (ITO) (45 nm) and dried to form a bulk heterojunction layer (300 nm) which was followed by a layer of ZnO (50 nm) and a layer of silver (60 nm). The device was annealed at 100° C. for 10 minutes and sealed to the substrate using a cover glass.

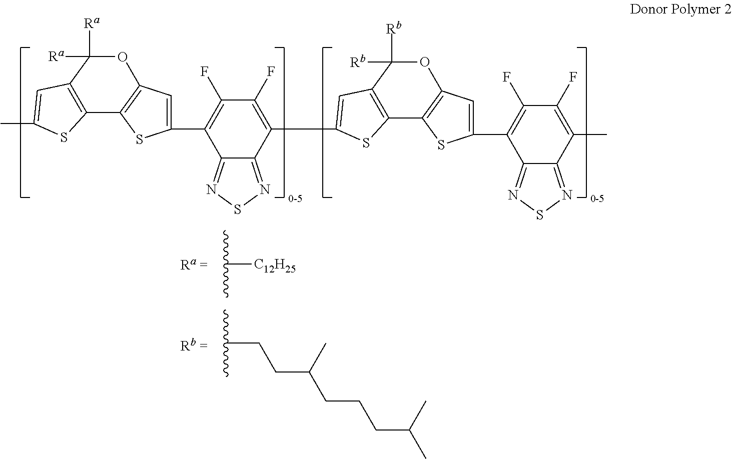

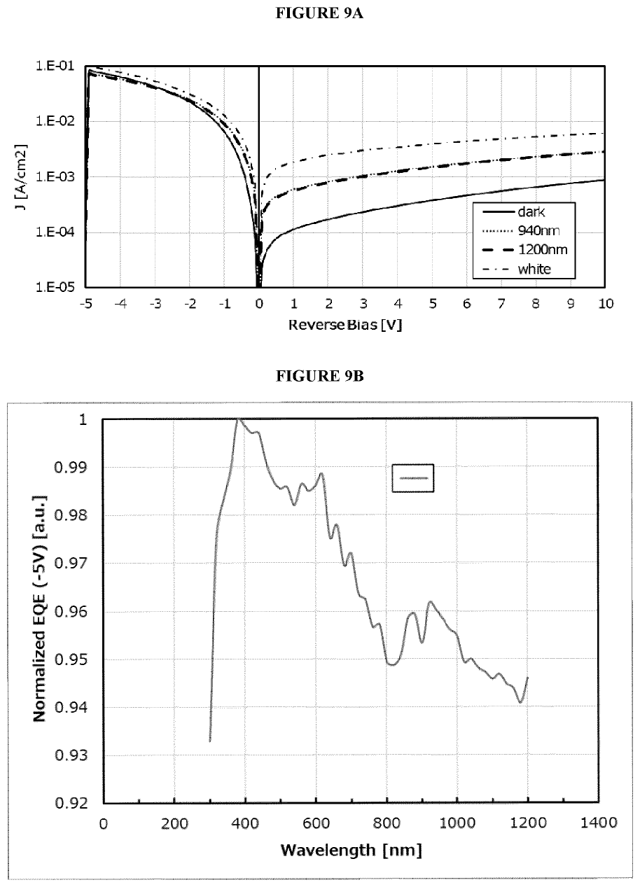

As shown in FIG. 9A, a response which is clearly distinguishable from dark current is observed at wavelengths of 940 nm, 1200 nm and under white light conditions upon application of a reverse bias.

As shown in FIG. 9B, a response is observed at wavelengths above 900 nm.

### Device Example 3

A device was prepared as described for Device Example 2 except that Compound Example 4 was used in place of Compound Example 3.

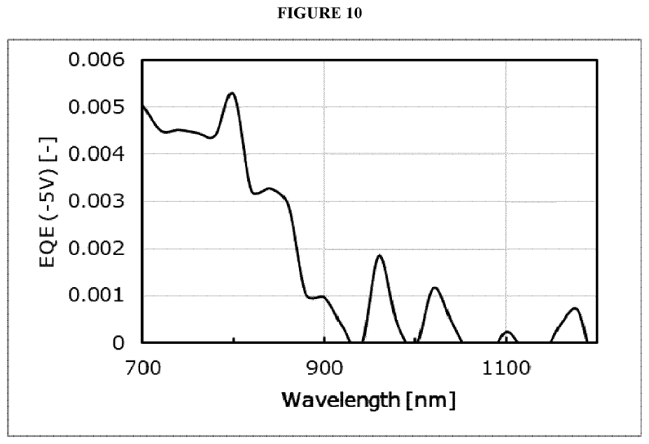

The external quantum efficiency for this device across a range of wavelengths at an applied bias of −5V is shown in FIG. 10.

### Device Example 4-6

Device Examples 4-6 were prepared as described for Device Example 1 except that Compound Examples 5-7, respectively, were used in place of Compound Example 1.

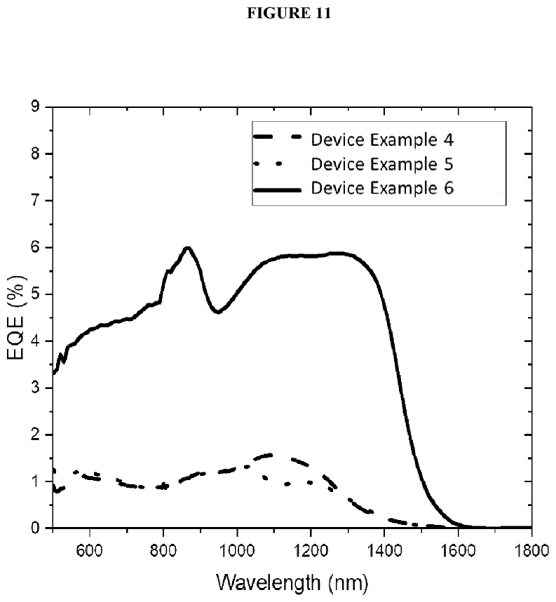

The external quantum efficiencies for these devices across a range of wavelengths at an applied bias of −5V is shown in FIG. 11.

### Modelling Examples (1)

All modelling as described in these examples was performed using Gaussian09 software available from Gaussian using Gaussian09 with B3LYP (functional).

HOMO and LUMO levels were modelled for individual donor and acceptor units. Results are set out in Tables 1-3

TABLE 1

| | HOMO/ | LUMO/ |
|---|---|---|
| Donor units D¹, D² | eV | eV |
| 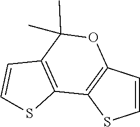 | −5.08 | −1.09 |
| 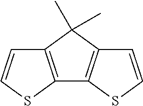 | −5.18 | −1.01 |
| 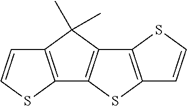 [SMILES: CC1(C)c2ccsc2-c2sc3ccsc3c21] | −5.06 | −1.25 |
| 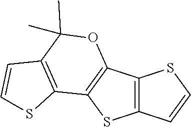 [SMILES: CC1(C)Oc2c(sc3ccsc23)-c2sccc21] | −4.97 | −1.32 |
| 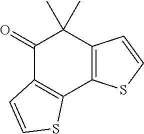 | −5.74 | −1.87 |
| 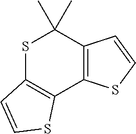 | −5.27 | −1.33 |

TABLE 2

| | HOMO/ | LUMO/ |
|---|---|---|
| Acceptor units A², A³ | eV | eV |
| 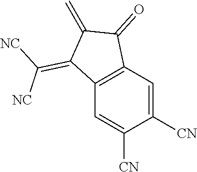 [SMILES: C=C1C(=O)c2cc(C#N)c(C#N)cc2C1=C(C#N)C#N] | −7.95 | −4.23 |
| 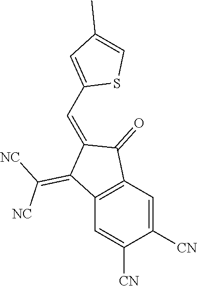 [SMILES: Cc1csc(/C=C2\C(=O)c3cc(C#N)c(C#N)cc3C2=C(C#N)C#N)c1] | −6.32 | −3.34 |
| 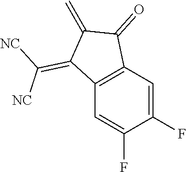 [SMILES: C=C1C(=O)c2cc(F)c(F)cc2C1=C(C#N)C#N] | −7.39 | −3.57 |

TABLE 3

| | HOMO/ | LUMO |
|---|---|---|
| Acceptor Unit A¹ | eV | /eV |
| 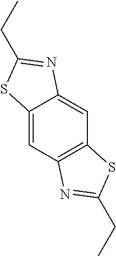 [SMILES: CCc1nc2cc3sc(CC)nc3cc2s1] | −6.02 | −1.25 |
| 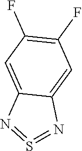 [SMILES: Fc1cc2c(cc1F)N=S=N2] | −6.95 | −2.60 |
| 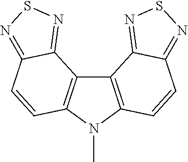 [SMILES: Cn1c2ccc3nsnc3c2c2c3nsnc3ccc21] | −5.72 | −2.15 |
| 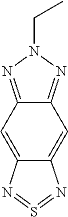 | −5.80 | −2.82 |
| 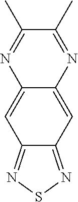 | −6.17 | −2.93 |
| 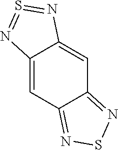 [SMILES: c1c2c(cc3nsnc13)N=S=N2] | −6.20 | −3.56 |
| 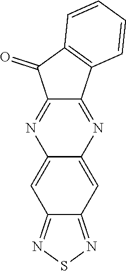 [SMILES: O=C1c2ccccc2-c2nc3cc4nsnc4cc3nc21] | −6.44 | −3.36 |

Acceptor units A¹ preferably have a modelled LUMO of at least 2.9 eV or at least 3.0 eV from vacuum level.

HOMO and LUMO levels were modelled for compounds of formula (I) in which there is no bridge between A² and D¹ (z1=0) or between A³ and D² (z2=0).

Results are set out in Table 4. Sif corresponds to oscillator strength of the transition from S1 (predicting absorption intensity), Eopt is the modelled optical gap.

TABLE 4

| | HOMO/ | LUMO/ | Eg/ | | Eopt/ |
|---|---|---|---|---|---|
| Structure/ID | eV | eV | eV | S1f | nm |
| 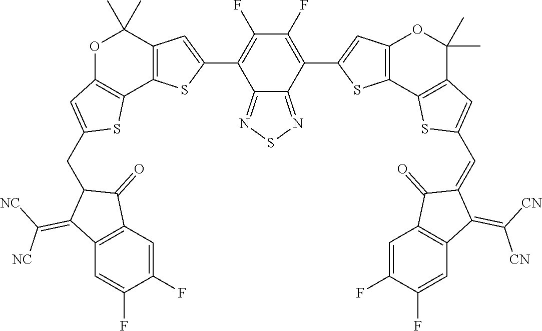 [SMILES: CC1(C)Oc2cc(-c3c(F)c(F)c(-c4cc5c(s4)-c4sc(CC6C(=O)c7cc(F)c(F)cc7C6=C(C#N)C#N)cc4OC5(C)C)c4nsnc34)sc2-c2sc(/C=C3\C(=O)c4cc(F)c(F)cc4C3=C(C#N)C#N)cc21] | −5.49 | −3.74 | 708 | 2.11 | 802 |
| 405 | | | | | |
| Comparative | | | | | |
| 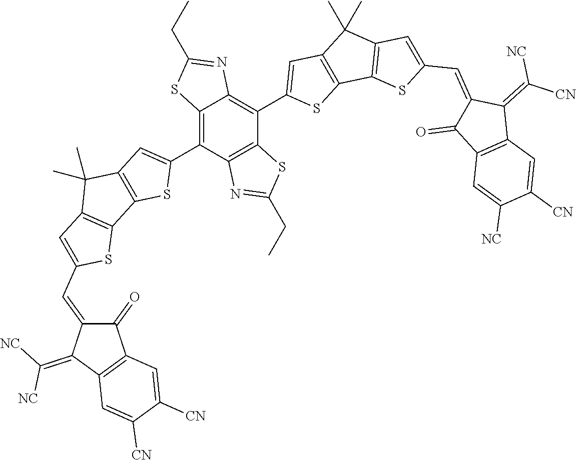 [SMILES: CCc1nc2c(-c3cc4c(s3)-c3sc(/C=C5\C(=O)c6cc(C#N)c(C#N)cc6C5=C(C#N)C#N)cc3C4(C)C)c3sc(CC)nc3c(-c3cc4c(s3)-c3sc(/C=C5\C(=O)c6cc(C#N)c(C#N)cc6C5=C(C#N)C#N)cc3C4(C)C)c2s1] | −5.80 | −3.85 | 634 | 2.47 | 711 |
| Comparative 638 | | | | | |
| 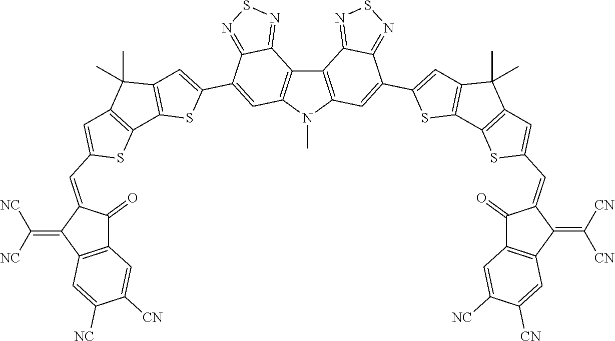 [SMILES: Cn1c2cc(-c3cc4c(s3)-c3sc(/C=C5\C(=O)c6cc(C#N)c(C#N)cc6C5=C(C#N)C#N)cc3C4(C)C)c3nsnc3c2c2c3nsnc3c(-c3cc4c(s3)-c3sc(/C=C5\C(=O)c6cc(C#N)c(C#N)cc6C5=C(C#N)C#N)cc3C4(C)C)cc21] | −5.71 | −3.91 | 688 | 2.55 | 777 |
| Comparative 641 | | | | | |
| 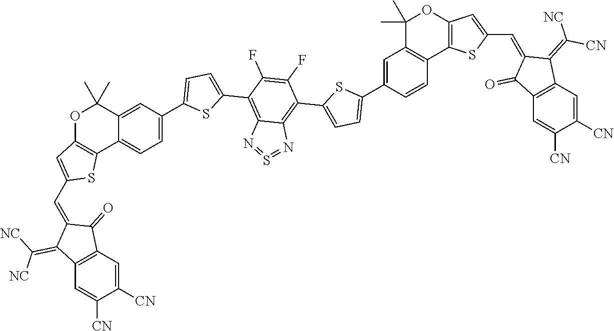 [SMILES: CC1(C)Oc2cc(/C=C3\C(=O)c4cc(C#N)c(C#N)cc4C3=C(C#N)C#N)sc2-c2ccc(-c3ccc(-c4c(F)c(F)c(-c5ccc(-c6ccc7c(c6)C(C)(C)Oc6cc(/C=C8\C(=O)c9cc(C#N)c(C#N)cc9C8=C(C#N)C#N)sc6-7)s5)c5c4N=S=N5)s3)cc21] | −5.72 | −3.87 | 669 | 2.41 | 760 |
| Comparative 642 | | | | | |
| 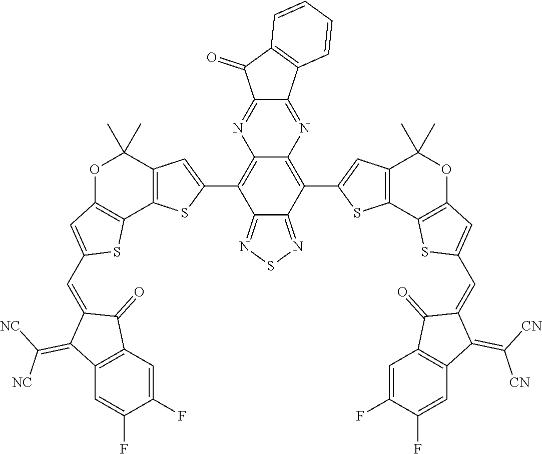 [SMILES: CC1(C)Oc2cc(/C=C3\C(=O)c4cc(F)c(F)cc4C3=C(C#N)C#N)sc2-c2sc(-c3c4nsnc4c(-c4cc5c(s4)-c4sc(/C=C6\C(=O)c7cc(F)c(F)cc7C6=C(C#N)C#N)cc4OC5(C)C)c4nc5c(nc34)C(=O)c3ccccc3-5)cc21] | −5.30 | −4.07 | 1008 | 1.32 | 1142 |
| 568 | | | | | |
| 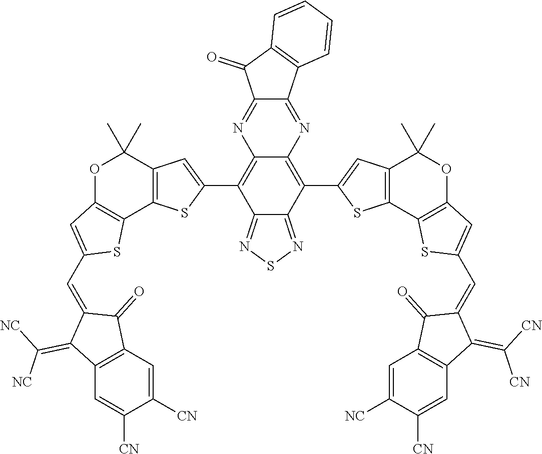 [SMILES: CC1(C)Oc2cc(/C=C3\C(=O)c4cc(C#N)c(C#N)cc4C3=C(C#N)C#N)sc2-c2sc(-c3c4nsnc4c(-c4cc5c(s4)-c4sc(/C=C6\C(=O)c7cc(C#N)c(C#N)cc7C6=C(C#N)C#N)cc4OC5(C)C)c4nc5c(nc34)C(=O)c3ccccc3-5)cc21] | −5.62 | −4.38 | 1000 | 1.32 | 1123 |
| 566 | | | | | |
| 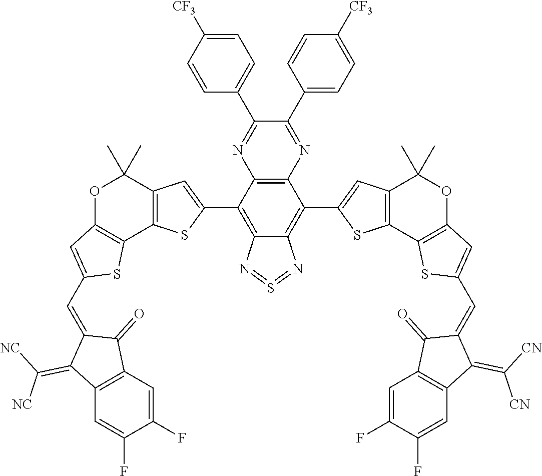 [SMILES: CC1(C)Oc2cc(/C=C3\C(=O)c4cc(F)c(F)cc4C3=C(C#N)C#N)sc2-c2sc(-c3c4c(c(-c5cc6c(s5)-c5sc(/C=C7\C(=O)c8cc(F)c(F)cc8C7=C(C#N)C#N)cc5OC6(C)C)c5nc(-c6ccc(C(F)(F)F)cc6)c(-c6ccc(C(F)(F)F)cc6)nc35)N=S=N4)cc21] | −5.31 | −4.11 | 1027 | 1.27 | 1133 |
| 492 | | | | | |
| 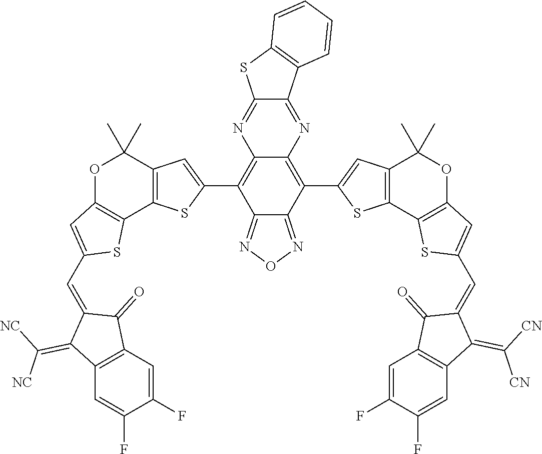 [SMILES: CC1(C)Oc2cc(/C=C3\C(=O)c4cc(F)c(F)cc4C3=C(C#N)C#N)sc2-c2sc(-c3c4nonc4c(-c4cc5c(s4)-c4sc(/C=C6\C(=O)c7cc(F)c(F)cc7C6=C(C#N)C#N)cc4OC5(C)C)c4nc5c(nc34)sc3ccccc35)cc21] | −5.28 | −4.08 | 1036 | 1.38 | 1114 |
| 489 | | | | | |
| 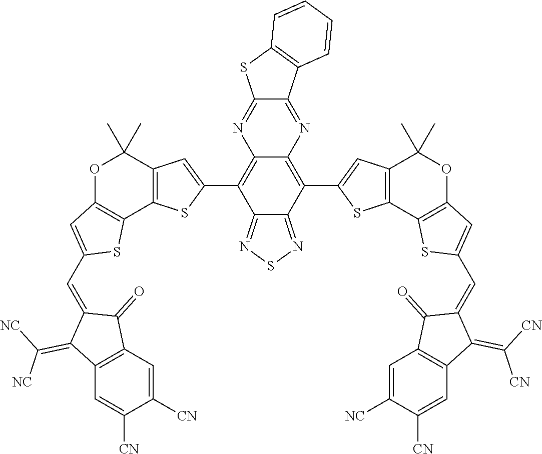 [SMILES: CC1(C)Oc2cc(/C=C3\C(=O)c4cc(C#N)c(C#N)cc4C3=C(C#N)C#N)sc2-c2sc(-c3c4nsnc4c(-c4cc5c(s4)-c4sc(/C=C6\C(=O)c7cc(C#N)c(C#N)cc7C6=C(C#N)C#N)cc4OC5(C)C)c4nc5c(nc34)sc3ccccc35)cc21] | −5.55 | −4.34 | 1018 | 1.45 | 1118 |
| 490 | | | | | |
| 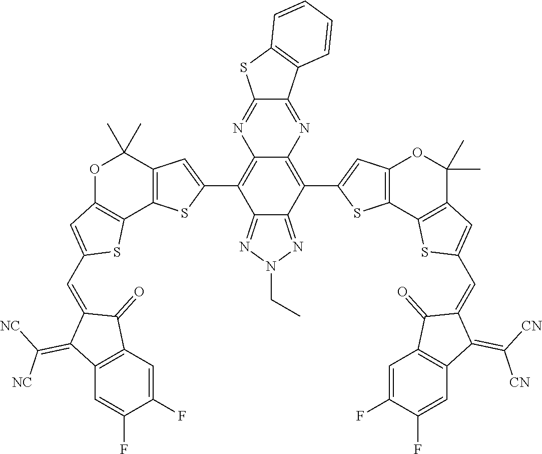 [SMILES: CCn1nc2c(-c3cc4c(s3)-c3sc(/C=C5\C(=O)c6cc(F)c(F)cc6C5=C(C#N)C#N)cc3C(C)(C)O4)c3nc4c(nc3c(-c3cc5c(s3)-c3sc(/C=C6\C(=O)c7cc(F)c(F)cc7C6=C(C#N)C#N)cc3OC5(C)C)c2n1)sc1ccccc14] | −5.20 | −3.73 | 842 | 1.83 | 927 |
| 482 | | | | | |
| 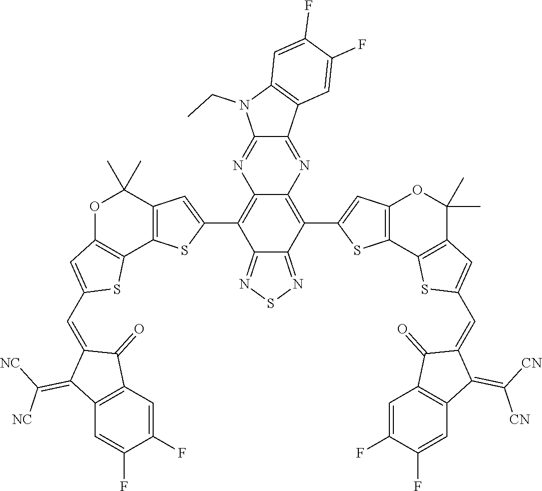 [SMILES: CCn1c2cc(F)c(F)cc2c2nc3c(-c4cc5c(s4)-c4sc(/C=C6\C(=O)c7cc(F)c(F)cc7C6=C(C#N)C#N)cc4C(C)(C)O5)c4nsnc4c(-c4cc5c(s4)-c4sc(/C=C6\C(=O)c7cc(F)c(F)cc7C6=C(C#N)C#N)cc4OC5(C)C)c3nc21] | −5.23 | −3.94 | 958 | 1.30 | 1076 |
| 486 | | | | | |
| 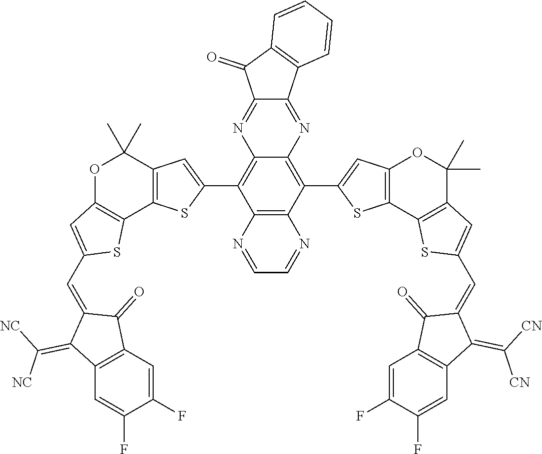 [SMILES: CC1(C)Oc2cc(-c3c4nccnc4c(-c4cc5c(s4)-c4sc(/C=C6\C(=O)c7cc(F)c(F)cc7C6=C(C#N)C#N)cc4OC5(C)C)c4nc5c(nc34)-c3ccccc3C5=O)sc2-c2sc(/C=C3\C(=O)c4cc(F)c(F)cc4C3=C(C#N)C#N)cc21] | −5.31 | −3.95 | 911 | 1.13 | 1060 |
| 476 | | | | | |
| 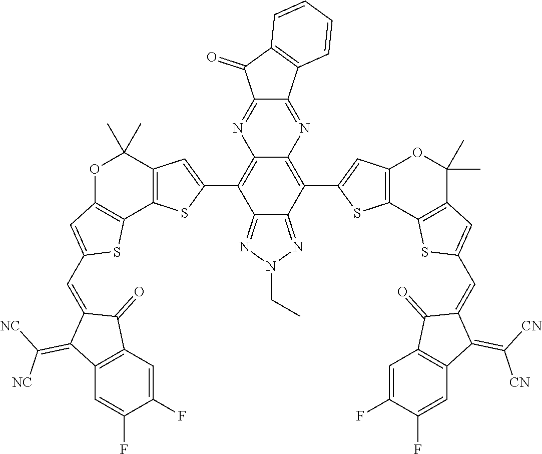 [SMILES: CCn1nc2c(-c3cc4c(s3)-c3sc(/C=C5\C(=O)c6cc(F)c(F)cc6C5=C(C#N)C#N)cc3C(C)(C)O4)c3nc4c(nc3c(-c3cc5c(s3)-c3sc(/C=C6\C(=O)c7cc(F)c(F)cc7C6=C(C#N)C#N)cc3OC5(C)C)c2n1)C(=O)c1ccccc1-4] | −5.26 | −3.79 | 843 | 1.51 | 949 |
| 477 | | | | | |
| 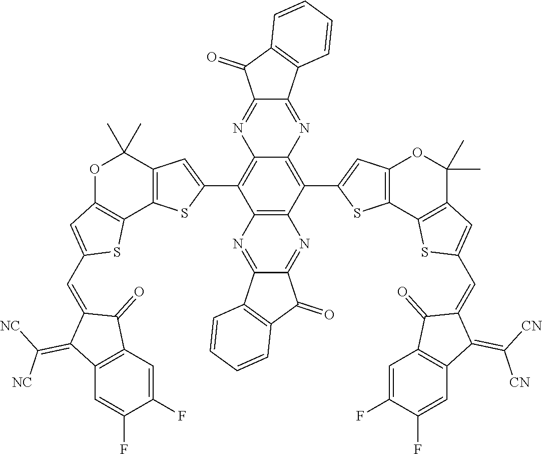 [SMILES: CC1(C)Oc2cc(-c3c4nc5c(nc4c(-c4cc6c(s4)-c4sc(/C=C7\C(=O)c8cc(F)c(F)cc8C7=C(C#N)C#N)cc4OC6(C)C)c4nc6c(nc34)c(=O)c3ccccc36)c(=O)c3ccccc35)sc2-c2sc(/C=C3\C(=O)c4cc(F)c(F)cc4C3=C(C#N)C#N)cc21] | −5.30 | −3.98 | 937 | 0.89 | 1124 |
| 479 | | | | | |
| 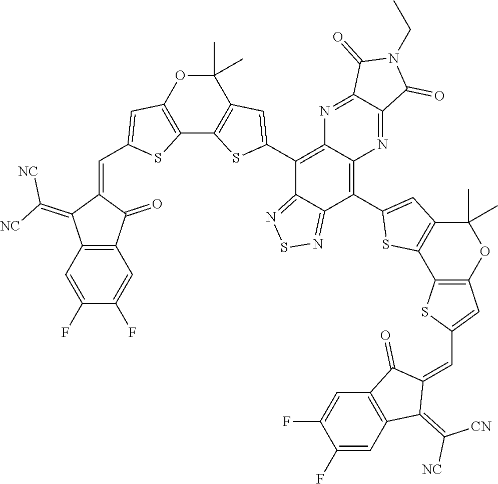 [SMILES: CCN1C(=O)c2nc3c(-c4cc5c(s4)-c4sc(/C=C6\C(=O)c7cc(F)c(F)cc7C6=C(C#N)C#N)cc4OC5(C)C)c4nsnc4c(-c4cc5c(s4)-c4sc(/C=C6\C(=O)c7cc(F)c(F)cc7C6=C(C#N)C#N)cc4OC5(C)C)c3nc2C1=O] | −5.34 | −4.24 | 1120 | 1.12 | 1242 |
| 541 | | | | | |
| 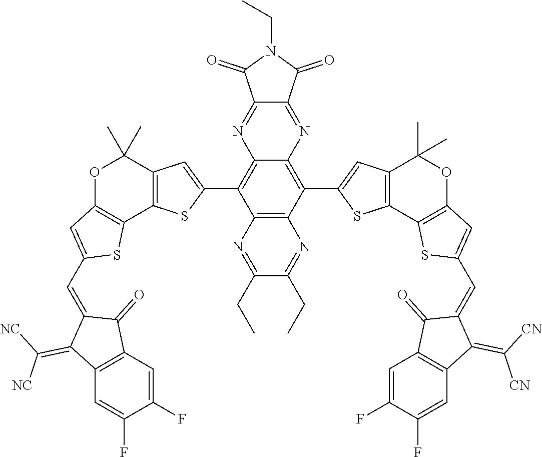 [SMILES: CCc1nc2c(-c3cc4c(s3)-c3sc(/C=C5\C(=O)c6cc(F)c(F)cc6C5=C(C#N)C#N)cc3OC4(C)C)c3nc4c(nc3c(-c3cc5c(s3)-c3sc(/C=C6\C(=O)c7cc(F)c(F)cc7C6=C(C#N)C#N)cc3OC5(C)C)c2nc1CC)C(=O)N(CC)C4=O] | −5.30 | −3.96 | 928 | 1.25 | 1074 |
| 542 | | | | | |
| 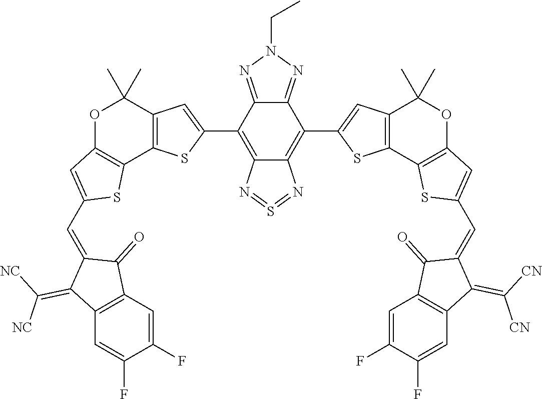 [SMILES: CCn1nc2c(-c3cc4c(s3)-c3sc(/C=C5\C(=O)c6cc(F)c(F)cc6C5=C(C#N)C#N)cc3OC4(C)C)c3c(c(-c4cc5c(s4)-c4sc(/C=C6\C(=O)c7cc(F)c(F)cc7C6=C(C#N)C#N)cc4OC5(C)C)c2n1)N=S=N3] | −5.16 | −3.90 | 989 | 1.90 | 1032 |
| 491 | | | | | |
| 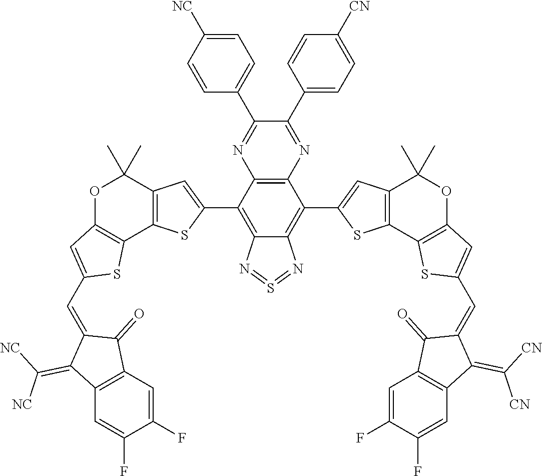 [SMILES: CC1(C)Oc2cc(/C=C3\C(=O)c4cc(F)c(F)cc4C3=C(C#N)C#N)sc2-c2sc(-c3c4c(c(-c5cc6c(s5)-c5sc(/C=C7\C(=O)c8cc(F)c(F)cc8C7=C(C#N)C#N)cc5OC6(C)C)c5nc(-c6ccc(C#N)cc6)c(-c6ccc(C#N)cc6)nc35)N=S=N4)cc21] | −5.39 | −4.21 | 1046 | 1.20 | 1157 |
| 493 | | | | | |
| 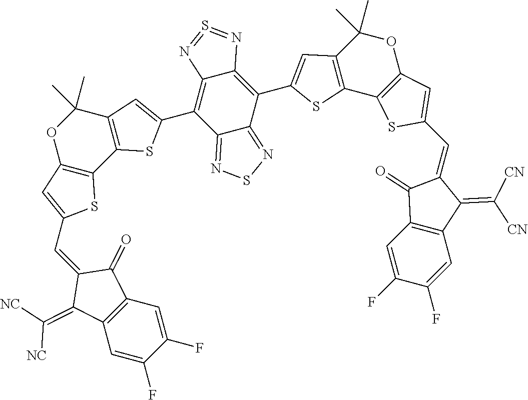 [SMILES: CC1(C)Oc2cc(/C=C3\C(=O)c4cc(F)c(F)cc4C3=C(C#N)C#N)sc2-c2sc(-c3c4c(c(-c5cc6c(s5)-c5sc(/C=C7\C(=O)c8cc(F)c(F)cc8C7=C(C#N)C#N)cc5OC6(C)C)c5nsnc35)N=S=N4)cc21] | −5.22 | −4.22 | 1239 | 1.20 | 1281 |
| 504 | | | | | |
| 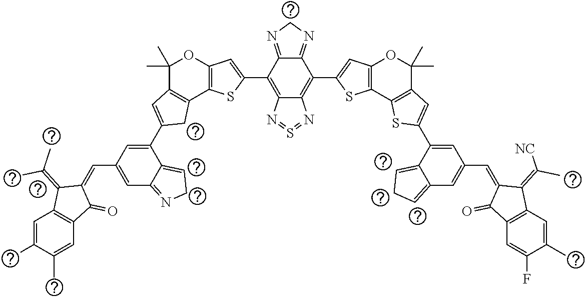 | −4.67 | −3.76 | 0.93 | | 1288 |

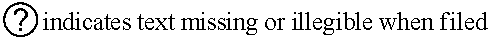

### Modelling Examples (2)

HOMO and LUMO levels were modelled for compounds of formula (I) in which there is a bridge between A² and D¹ (z1=1) and between A³ and D² (z2=1).

| Structure/ID | HOMO/eV | LUMO/eV | Eg/eV | S1f | Eopt/nm |
|---|---|---|---|---|---|
| 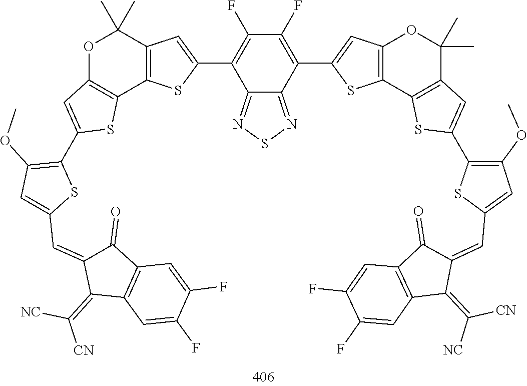 [SMILES: COc1cc(/C=C2\C(=O)c3cc(F)c(F)cc3C2=C(C#N)C#N)sc1-c1cc2c(s1)-c1sc(-c3c(F)c(F)c(-c4cc5c(s4)-c4sc(-c6sc(/C=C7\C(=O)c8cc(F)c(F)cc8C7=C(C#N)C#N)cc6OC)cc4C(C)(C)O5)c4nsnc34)cc1C(C)(C)O2] | −5.02 | −3.51 | 821 | 2.03 | 937 |
| 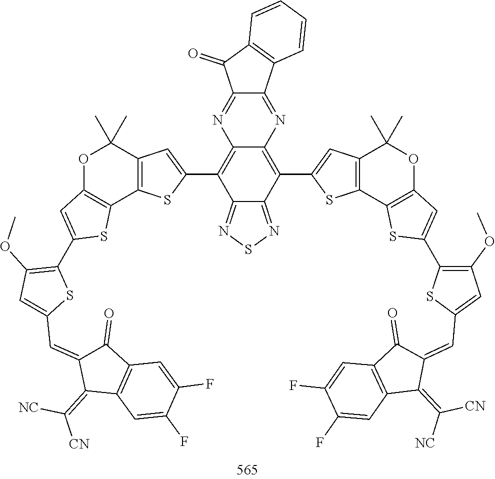 [SMILES: COc1cc(/C=C2\C(=O)c3cc(F)c(F)cc3C2=C(C#N)C#N)sc1-c1cc2c(s1)-c1sc(-c3c4nsnc4c(-c4cc5c(s4)-c4sc(-c6sc(/C=C7\C(=O)c8cc(F)c(F)cc8C7=C(C#N)C#N)cc6OC)cc4OC5(C)C)c4nc5c(nc34)C(=O)c3ccccc3-5)cc1C(C)(C)O2] | −4.84 | −3.78 | 1165 | 1.23 | 1329 |
| 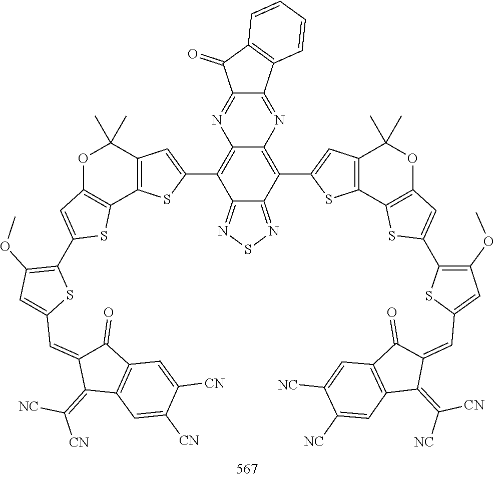 [SMILES: COc1cc(/C=C2\C(=O)c3cc(C#N)c(C#N)cc3C2=C(C#N)C#N)sc1-c1cc2c(s1)-c1sc(-c3c4nsnc4c(-c4cc5c(s4)-c4sc(-c6sc(/C=C7\C(=O)c8cc(C#N)c(C#N)cc8C7=C(C#N)C#N)cc6OC)cc4OC5(C)C)c4nc5c(nc34)C(=O)c3ccccc3-5)cc1C(C)(C)O2] | −5.11 | −4.05 | 1180 | 1.41 | 1327 |
| 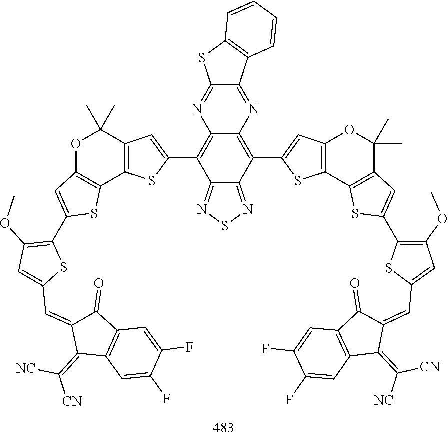 [SMILES: COc1cc(/C=C2\C(=O)c3cc(F)c(F)cc3C2=C(C#N)C#N)sc1-c1cc2c(s1)-c1sc(-c3c4nsnc4c(-c4cc5c(s4)-c4sc(-c6sc(/C=C7\C(=O)c8cc(F)c(F)cc8C7=C(C#N)C#N)cc6OC)cc4C(C)(C)O5)c4nc5c(nc34)sc3ccccc35)cc1C(C)(C)O2] | −5.08 | −3.61 | 1037 | 0.66 | 1238 |

1. A compound of formula (I): 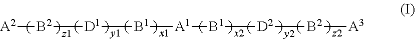
wherein:
D¹ and D² independently in each occurrence is an electron-donating group;
A² and A³ are each independently an electron-accepting group;
B¹ and B² in each occurrence are independently a bridging group;
x1 and x2 are each independently 0, 1, 2 or 3;
y1 and y2 are each independently at least 1;
z1 and z2 are each independently 0, 1, 2 or 3; and
A¹ is a group of formula (II): 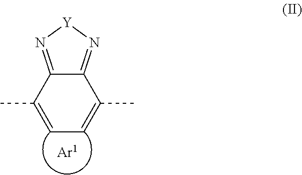
wherein:
Ar¹ is an aromatic or heteroaromatic group; and
Y is O, S, NR⁴ or R¹—C═C—R¹ wherein R¹ in each occurrence is independently H or a substituent wherein two substituents R¹ may be linked to form a monocyclic or polycyclic ring and R⁴ is H or a substituent.

2. The compound according to claim 1 wherein the group of formula (II) has formula (IIa):

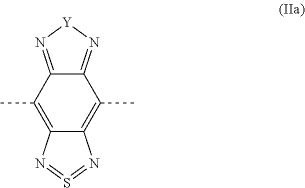

3. The compound according to claim 1 wherein the group of formula (II) has formula (IIb):

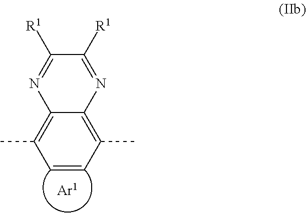

4. The compound according to claim 3 wherein the two R¹ groups are not linked.

5. The compound according to claim 4 wherein each R¹ is independently selected from H; F; CN; NO₂; C₁₋₂₀ alkyl wherein one or more non-adjacent C atoms may be replaced with O, S, CO, COO, NR⁴, PR⁴, or Si(R³)₂ and one or more H atoms may be replaced with F; and aryl or heteroaryl which may be unsubstituted or substituted with one or more substituents, wherein R³ and R⁴ are each independently H or a substituent.

6. The compound according to claim 3 wherein the two R¹ groups are linked.

7. The compound according to claim 6 wherein the compound of formula (IIb) has formula (IIb-1) or (IIb-2): 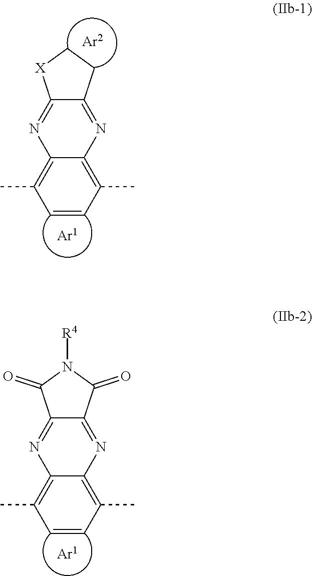
wherein
Ar² is an aromatic or heteroaromatic group which is unsubstituted or substituted with one or more substituents; and
X is selected from O, S, SO₂, NR⁴, PR⁴, C(R³)₂, Si(R³)₂ C═O, C═S and C═C(R⁵)₂ wherein R³ and R⁴ independently in each occurrence are selected from H and a substituent and R⁵ independently in each occurrence is an electron-withdrawing group.

8. The compound according to claim 7 wherein Ar² is benzene which is unsubstituted or substituted with one or more substituents.

9. (canceled)

10. The compound according to claim 1 wherein at least one of z1 and z2 is at least 1 and B² in each occurrence is independently selected from vinylene, arylene, heteroarylene, arylenevinylene and heteroarylenevinylene, each of which is unsubstituted or substituted with one or more substituents.

11. The compound according to claim 1 wherein D1 and D2 are each independently selected from units of formulae (VIIa)-(VIIp): 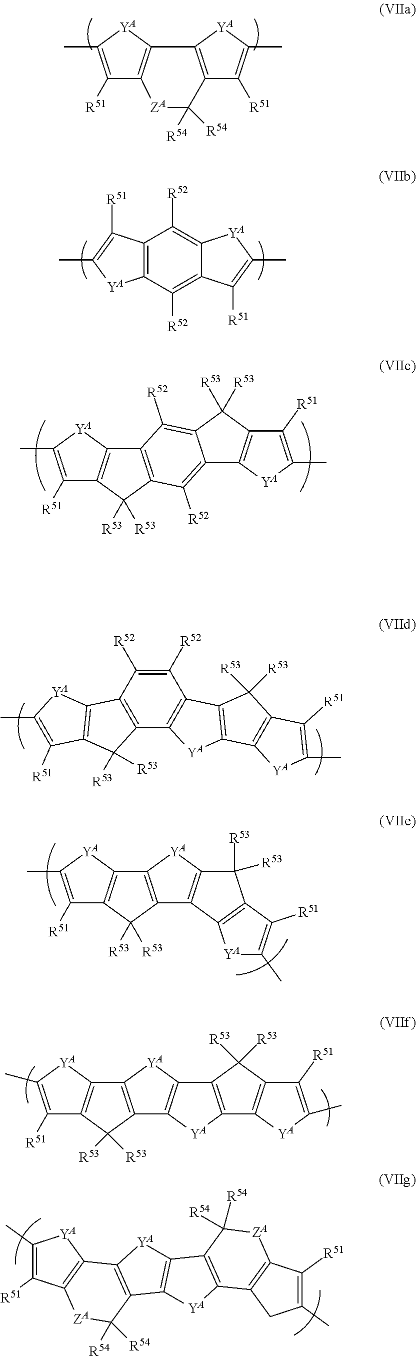 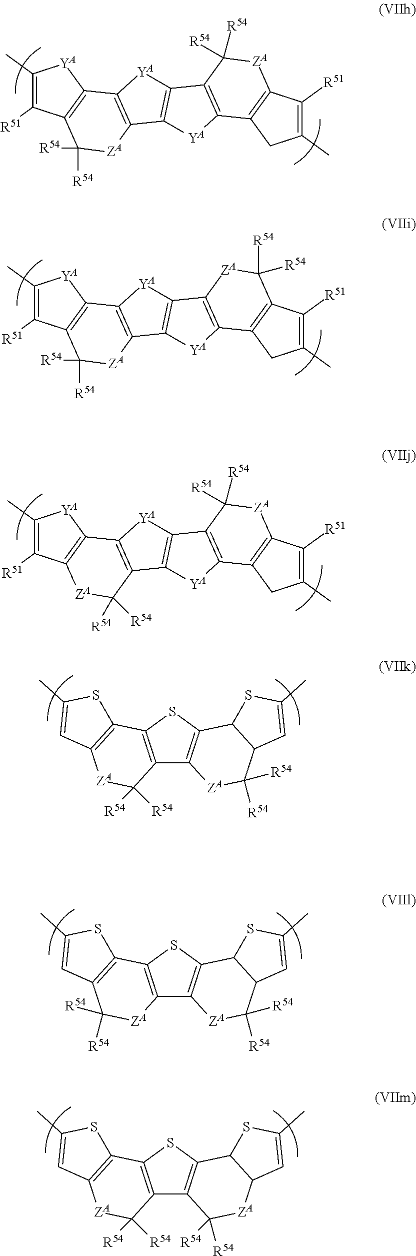 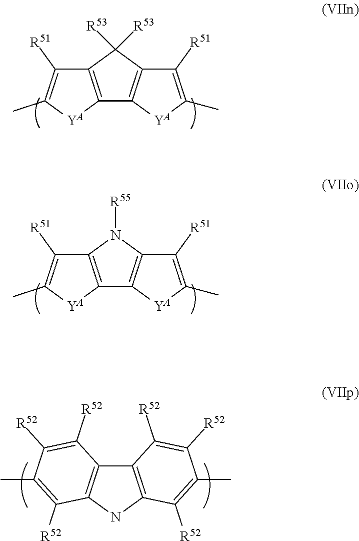
wherein Y^{A} in each occurrence is independently O, S or NR⁵⁵, Z^{A} in each occurrence is O, S, NR⁵⁵ or C(R⁵⁴)₂; R⁵¹, R⁵² R⁵⁴ and R⁵⁵ independently in each occurrence is H or a substituent;
and R⁵³ independently in each occurrence is a substituent.

12. The compound according to claim 1 wherein at least one of A² and A³ comprises a non-aromatic carbon-carbon double bond and a carbon atom of the carbon-carbon double bond is bound directly to D¹ or D² or, if present, to B².

13. (canceled)

14. The compound according to claim 12 wherein at least one A is a group of formula (IIIa-1): 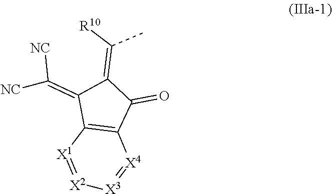
wherein:
each X¹—X⁴ is independently CR¹² or N wherein R¹² in each occurrence is H or a substituent selected from C₁₋₂₀ hydrocarbyl and an electron withdrawing group.

15. The compound according to claim 1 wherein the polymer has an absorption peak of greater than 900 nm.

16. (canceled)

17. (canceled)

18. A composition comprising an electron-donating material and an electron-accepting material wherein the electron accepting material is a compound according to claim 1.

19. An organic electronic device comprising an active layer comprising a compound according to claim 1.

20. An organic electronic device comprising an active layer comprising a compound according to claim 1 wherein the organic electronic device is an organic photoresponsive device comprising a bulk heterojunction layer disposed between an anode and a cathode and wherein the bulk heterojunction layer comprises an electron-donating material and an electron-accepting material wherein the electron accepting material is a compound according to claim 1.

21. An organic electronic device according to claim 20 wherein the organic photoresponsive device is an organic photodetector.

22. A photosensor comprising a light source and an organic photodetector according to claim 21, wherein the photosensor is configured to detect light emitted from the light source.

23. The photosensor according to claim 22, wherein the light source emits light having a peak wavelength of greater than 900 nm.

24. A formulation comprising a compound according to claim 1 dissolved or dispersed in one or more solvents.

25. (canceled)
